(19) United States
(12) Patent Application Publication
(10) Pub. No.: US 2020/0157541 A1
(43) Pub. Date: May 21, 2020

(54) EXOSOME LOADED THERAPEUTICS FOR THE TREATMENT OF NON-ALCOHOLIC STEATOHEPATITIS, DIABETES MELLITUS TYPE 1 AND TYPE 2, ATHEROSCLEROTIC CARDIOVASCULAR DISEASE, AND ALPHA 1 ANTITRYPSIN DEFICIENCY

(71) Applicant: Exosome Therapeutics, Inc., San Diego, CA (US)

(72) Inventors: Gerardo Rodriguez-Araujo, San Diego, CA (US); Stephen R. Puckett, Sr., Charlotte, NC (US); Stephen R. Puckett, Jr., Charolotte, NC (US); Mitchell W. Puckett, Charolotte, NC (US)

(73) Assignee: Exosome Therapeutics, Inc., San Diego, CA (US)

(21) Appl. No.: 16/688,954

(22) Filed: Nov. 19, 2019

Related U.S. Application Data
(63) Continuation-in-part of application No. 16/591,502, filed on Oct. 2, 2019.
Application No. 16/591,502 is a continuation-in-part of application No. 16/591,483, filed on Oct. 2, 2019.
(60) Provisional application No. 62/769,123, filed on Nov. 19, 2018.
(60) Provisional application No. 62/769,774, filed on Nov. 20, 2018.
(60) Provisional application No. 62/769,711, filed on Nov. 20, 2018.
(60) Provisional application No. 62/770,640, filed on Nov. 21, 2018.

(51) Int. Cl. C12N 15/113

(52) U.S. Cl. CPC C12N 15/113; C12N 2310/20; C12N 2800/80; C12N 2320/32; C12N 2310/14

(57) ABSTRACT

A composition for delivering a cargo to the cytoplasm of a cell, wherein the cargo treats Non-Alcoholic Steatohepatitis, Non-Alcoholic Fatty Liver Disease, Diabetes Mellitus Type 1 and Type 2, Atherosclerotic Cardiovascular Disease, and Alpha 1 Antitrypsin Deficiency. In another embodiment, the composition comprises: an exosome; cargo located inside the exosome, wherein the cargo comprises short interference RNA (siRNA) that depletes sodium-glucose linked transporter active sites. In another embodiment, the composition comprises: an exosome; cargo location inside the exosome, wherein the cargo corrects the missense SERPINA1 mutation from ‘Z’ to ‘M’.

### CROSS-REFERENCE TO RELATED APPLICATION

The present application is a continuation-in-part of, and claims priority to, U.S. patent application Ser. No. 16/591,502 filed Oct. 2, 2019, entitled “EXOSOME LOADED THERAPEUTICS FOR TREATING SICKLE CELL DISEASE,” which claims priority to U.S. Provisional Patent Application Nos. 62/740,396 filed Oct. 2, 2018, entitled “METHODS OF PRODUCING cGMP GRADE AND RESEARCH ONLY GRADE AUTOLOGOUS AND ALLOGENIC EXOSOMES AS CARRIERS FOR THERAPEUTIC COMPOUNDS FOR USE IN HUMANS AND IN PRECLINICAL STUDIES IN ANIMALS;” and 62/769,123 filed Nov. 19, 2018, entitled “cGMP Exosome Loaded Therapeutics for Sickle Cell Disease (SCD), SCD Anemia and its Associated Complications Reverting the Single Gene Mutation from Thymine to Adenine in the SNP rs334 in the Chromosome 11 and/or Reestablishing Normal Wild Type Healthy Genotype T>A (normal Adenine phenotype) to Produce Adult Beta Globin for Use in Humans and in Preclinical Studies in Animals,” the entire disclosures or which are incorporated by reference herein.

The present application also claims priority to, U.S. patent application Ser. No. 16/591,483 filed Oct. 2, 2019, entitled “COMPOSITIONS AND METHODS FOR PRODUCING EXOSOME LOADED THERAPEUTICS FOR TREATING CARDIOVASCULAR DISEASE,” which claims priority to U.S. Provisional Patent Application Nos. 62/740,396 filed Oct. 2, 2018, entitled “METHODS OF PRODUCING cGMP GRADE AND RESEARCH ONLY GRADE AUTOLOGOUS AND ALLOGENIC EXOSOMES AS CARRIERS FOR THERAPEUTIC COMPOUNDS FOR USE IN HUMANS AND IN PRECLINICAL STUDIES IN ANIMALS;” 62/740,382 filed Oct. 2, 2018, entitled “cGMP GRADE AND RESEARCH ONLY GRADE AUTOLOGOUS EXOSOMES FOR THE PROMOTION (USING VASCULAR ENDOTHELIAL GROWN FACTOR 1, 2 AND 3) OR REPRESSION OF ANGIOGENESIS (ANTI-MIR-214) FOR USE IN HUMANS AND IN PRECLINICAL STUDIES IN ANIMALS;” and 62/740,391 filed Oct. 2, 2018, “cGMP GRADE AND RESEARCH ONLY GRADE AUTOLOGOUS EXOSOMES FOR THE PRIMARY AND/OR SECONDARY PREVENTION OF CARDIOVASCULAR DISEASE (CVD), INFLAMMATION AND THEIR ASSOCIATED COMPLICATIONS USING PCSK-9, IL-1B, IL4 AND 13 AS THERAPEUTIC TARGETS FOR USE IN HUMANS AND IN PRECLINICAL STUDIES IN ANIMALS,” the entire disclosures of which are incorporated by reference herein

The present application further claims priority to U.S. Provisional Patent Application Nos. 62/769,123 filed Nov. 19, 2018, entitled “cGMP Exosome Loaded Therapeutics for Sickle Cell Disease (SCD), SCD Anemia and its Associated Complications Reverting the Single Gene Mutation from Thymine to Adenine in the SNP rs334 in the Chromosome 11 and/or Reestablishing Normal Wild Type Healthy Genotype T>A (normal Adenine phenotype) to Produce Adult Beta Globin for Use in Humans and in Preclinical Studies in Animals;” 62/770,640 filed Nov. 21, 2018, entitled “CGMP EXOSOME LOADED THERAPEUTICS FOR THE TREATMENT OF MULTIPLE ONCOLOGICAL DISORDERS AND THE METHODOLOGY TO DESIGN, PRODUCE AND MANUFACTURE EXOSOME-BASED CAR-T CELLS FOR USE IN HUMANS AND IN PRECLINICAL STUDIES IN ANIMALS,” 62/769,774 filed Nov. 20, 2018, entitled “cGMP Exosome Loaded Therapeutics Using Depletion and Self-production of Antibodies Against Sodium Glucose Like Transporters 1 and/or 2 for the Treatment of Diabetes Mellitus Type 1 and Type 2, Non Alcoholic Steatosis, Non Alcoholic Steatohepatitis, Atherosclerotic Cardiovascular Disease and Chronic Heart Failure with Low and Preserved Ejection Fraction for Use in Humans and in Preclinical Studies in Animals,” and 62/769,711 filed Nov. 20, 2018 entitled “cGMP Exosome Loaded Therapeutics to Correct SERPINA1 Mutation and to Reestablish Normal Physiological Alpha 1 Antitrypsin Levels for Primary and Secondary Prevention of Alpha 1 Antitrypsin Deficiency Related-Liver Disease (including Emphysema and Cirrhosis), Lung Function Deficiencies and Chronic Obstructive Pulmonary Disease (COPD) for Use in Humans and in Preclinical Studies in Animals,” the entire disclosures of which are incorporated by reference herein.

### BACKGROUND OF THE INVENTION

### 1. Field of Invention

This invention relates to extracellular vesicles for therapeutic delivery, and more specifically, to exosome compositions and methods of producing the same for treatment of Diabetes Mellitus Type 1 (T1DM) and Type 2 (T2DM), Non Alcoholic Steatosis/Fatty Liver Disease and Steatohepatitis (NAFLD and NASH respectively), Atherosclerotic Cardiovascular Disease (ASCVD), Chronic Heart Failure (CHF), and Alpha 1 Antitrypsin Deficiency (A1ATD).

### 2. Description of Related Art

The current invention provides better treatment options for a number of illnesses. One example of such an illness is Diabetes Mellitus. Diabetes Mellitus consists of a series of conditions and manifestations that relate to poor glucose metabolism in organisms including humans. One of the main characteristics of Diabetes Mellitus is the lack of insulin or insulin action (Olokoba A B, Obateru O A, Olokoba L B. Type 2 diabetes mellitus: a review of current trends. Oman Med J 2012; 27(4): 269-73). This results in an excess of circulating glucose or hyperglycemia, which induces cell and tissue damage at cytosolic and nuclear levels with the glycation and increase of gene expression of inflammatory cytokines inducing an oxidative stress environment within the cell (Saltiel A R, Olefsky J M. Inflammatory mechanisms linking obesity and metabolic disease. J Clin Invest 2017; 127(1): 1-4). The oxidative stress environment leads to the mounting of an inflammatory response that will deter healthy cell populations of important cells such as beta cells in pancreas (producers of insulin) as well as deterioration on the insulin sensitivity of the receptor for insulin in the periphery (Rodriguez-Araujo G, Nakagami H. Pathophysiology of cardiovascular disease in diabetes mellitus. Cardiovascular Endocrinology & Metabolism 2018; 7(1): 4-9; Reaven G M. Insulin resistance: the link between obesity and cardiovascular disease. Med Clin North Am 2011; 95(5): 875-92).

To better treat Diabetes Mellitus, a relatively new class of drugs has been developed in the last decade involving sodium glucose linked transporters (SGLT). Using SGLT inhibitors can prevent certain receptors from reabsorbing glucose, sodium, and water from the ultrafiltrate in kidneys. The two most well-known members of the SGLT family are SGLT1 and SGLT2. The SGLT2 function accounts for approximately 80% of the glucose, water, and sodium reabsorption by the nephron, while the SGLT1 function accounts for approximately the remaining 20%. Inhibiting SGLT2 receptors results in the inhibition of reabsorption of glucose, water, and sodium at 80%, while inhibiting both receptors can result in the inhibition of reabsorption of glucose, water, and sodium at or near 100% (Spatola L, Finazzi S, Angelini C, Dauriz M, Badalamenti S. SGLT1 and SGLT1 Inhibitors: A Role to Be Assessed in the Current Clinical Practice. Diabetes Ther 2018; 9(1): 427-30). These SGLT inhibitors however require daily dosing and the cost to manufacture the inhibitors is high. High cost of manufacturing results in a high cost to consumers, making the inhibitors unsuitable for use in chronic therapies in patients that have no insurance coverage or insurance coverage that does not cover such medication. A long lasting formulation, which is not just physiological, but less expensive and straightforward is necessary in order to offer the full benefits of this treatment adherence to patients with type 1 and type 2 Diabetes Mellitus (T1DM and T2DM, respectively).

Atherosclerotic cardiovascular disease (ASCVD) is another example of an illness in which the current invention can provide better treatment options. ASCVD consists of the narrowing of the arteries by a buildup of a mixture of oxidized low-density lipoprotein (LDL), inflammatory infiltrates, fibrosis, and calcification (Ference B A, Ginsberg H N, Graham I, et al. Low-density lipoproteins cause atherosclerotic cardiovascular disease. 1. Evidence from genetic, epidemiologic, and clinical studies. A consensus statement from the European Atherosclerosis Society Consensus Panel. Eur Heart J 2017; 38(32): 2459-72). ASCVD affects millions of patients worldwide and many of those patients also have Diabetes Mellitus or are at risk for Diabetes Mellitus. Severe occlusions or sub-occlusions of the arteries can precipitate ischemic attacks such as myocardial infarctions, strokes, lower or upper extremity gangrene leading to amputation, etc. Currently, the 2018 American Diabetes Association together with the American College of Cardiology recommend the use of SGLT2 inhibitors in patients that have ASCVD in addition to Diabetes Mellitus as there is significant evidence that these drugs significantly reduce the risk for cardiovascular death and hospitalization for urgent revascularization in these patients (Abdul-Ghani M, Del Prato S, Chilton R, DeFronzo R A. SGLT2 Inhibitors and Cardiovascular Risk: Lessons Learned From the EMPA-REG OUTCOME Study. Diabetes Care 2016; 39(5): 717-25; American Diabetes A. 8. Pharmacologic Approaches to Glycemic Treatment: Standards of Medical Care in Diabetes-2018. Diabetes Care 2018; 41(Suppl 1): S73-S85; Yancy C W, Januzzi J L, Jr., Allen L A, et al. 2017 ACC Expert Consensus Decision Pathway for Optimization of Heart Failure Treatment: Answers to 10 Pivotal Issues About Heart Failure With Reduced Ejection Fraction: A Report of the American College of Cardiology Task Force on Expert Consensus Decision Pathways. J Am Coll Cardiol 2018; 71(2): 201-30).

In patients with ASCVD, ischemic attacks in the heart, such as myocardial infarctions, can lead to the remodeling of the anatomy and structure of the heart inducing postischemic CHF. Postischemic CHF refers to problems or lack of contractility to pump the blood from the ventricles to the lung and periphery. Postischemic Heart Failure is the most common type of CHF. The current guidelines for the treatment of CHF recommends SGLT2 inhibitors to be used in patients with or without Diabetes Mellitus because the inhibitors reduce the number of CHF re-hospitalizations and overall cardiovascular death (Abdul-Ghani M, Del Prato S, Chilton R, DeFronzo R A. SGLT2 Inhibitors and Cardiovascular Risk: Lessons Learned From the EMPA-REG OUTCOME Study. Diabetes Care 2016; 39(5): 717-25; Neal B, Perkovic V, Mahaffey K W, et al. Canagliflozin and Cardiovascular and Renal Events in Type 2 Diabetes. N Engl J Med 2017; 377(7): 644-57). CHF reduced ejection fraction is typically considered for patients with left ventricular ejection fraction (LVEF) of less than 30-40%. Preserved ejection fraction refers to LVEF of >40% in CHF patients.

Non Alcoholic Steatosis/Fatty Liver Disease and Steatohepatitis (NAFLD and NASH, respectively) are other examples of illnesses in which the current invention can provide better treatment options. NAFLD and NASH are mostly asymptomatic and in some situations patients may present generalized pruritus or itching in the skin with a slight elevation of transaminases with no viral or other possible etiology. The incidence is estimated to be about 25% of the general population. It is also estimated that 25% of patients with NASH or ‘the inflammatory ballooning of hepatocytes’ will develop NASH cirrhosis in the coming decades. In fact, it is expected that by 2030 the first indication of liver transplant will shift from alcoholic to non-alcoholic/NASH cirrhosis. No current pharmacotherapy exists for this indication. The industry is currently developing multiple small molecules and biologicals that target one or several pathways such as lipogenic, fibrogenic or inflammatory pathways to stop progression or induce regression of this disease (Konerman M A, Jones J C, Harrison S A. Pharmacotherapy for NASH: Current and emerging. J Hepatol 2018; 68(2): 362-75). However, the field lacks a consensus in determining targets for pharmacotherapy. Drug stability and short-term effects are a major concern in establishing a chronic therapy in the patient population. As NAFLD and NASH are multi-node diseases, multiple targets are necessary for successful treatment.

Alpha 1 Antitrypsin Deficiency (A1ATD) is a monogenic disease consisting in a missense mutation of the SERPINA1 gene that results in deficiency of alpha 1 antitrypsin (AAT). AAT neutralizes enzymes released by granulocytes and is important in inflammation and pyrexia. M alleles are normal physiological isoforms that translate to normal functional AAT. S alleles or Z alleles codify for toxic variants of AAT causing liver injury. Its mutant Z isoform (point mutation Glu342 to Lysine (Lys) of SERPINA1 gene) accumulates in the endoplasmic reticulum of hepatocytes with cytotoxic effects leading to cirrhosis (Mahr A D, Edberg J C, Stone J H, et al. Alpha(1)-antitrypsin deficiency-related alleles Z and S and the risk of Wegener's granulomatosis. Arthritis Rheum 2010; 62(12): 3760-7).

The most common manifestations of A1ATD are respiratory problems, emphysema, and jaundice with symptoms starting early in children and young adults (Fregonese L, Stolk J. Hereditary alpha-1-antitrypsin deficiency and its clinical consequences. Orphanet J Rare Dis 2008; 3: 16). Smoking is an environmental factor that can trigger the phenotype of the disease and its complications. Complications of A1ATD are respiratory problems including emphysema, chronic obstructive pulmonary disease (COPD) and liver injury leading to the development of liver insufficiency and cirrhosis (Donato L J, Karras R M, Katzmann J A, Murray D L, Snyder M R. Quantitation of circulating wild-type alpha-1-antitrypsin in heterozygous carriers of the S and Z deficiency alleles. Respir Res 2015; 16: 96). Currently, no cure exists for A1ATD and currently available treatments include human recombinant AAT therapy (high costs), messenger RNA therapy (mRNA; instability of RNA and dosing problems), interference RNA (RNAi) using ARC-AAT (study terminated due to poor results), protease inhibitors and the use of adeno-associated virus (AAV) to transduce hepatocytes to produce human AAT.

In light of these challenges in the field, there still exists an unmet medical need for treatment of these diseases. The invention relates to using exosomes loaded with DNA plasmids and any of its forms with or without RNAi to confer a long-lasting effect of self-producing neutralizing antibodies against any or both of the isoforms of SGLT receptors. There is an opportunity for advanced gene therapies to be loaded into autologous exosomes of cGMP (current Good Manufacturing Practices) grade for treatment. The present therapeutics include proteins, small molecules, peptides, gene therapies, bioengineered RNAi, plasmid DNA, DNA correction and modification materials, and any equivalent materials. The present method of using the therapeutics includes using cGMP grade exosomes as carriers of cGMP grade therapeutics. The present invention relates to exosome compositions and the methods of using the compositions. Wherein the compositions have superior quality and potency compared to the current biologicals.

Exosome assembly is a complex process. Researchers in the field are currently using viral vectors instead of exosomes. The invented method, for exosome loading, is superior in potency and efficacy as compared to any other method currently available. Further, the invented therapeutics have particularly high potency and efficacy. Exosomes are highly effective, virus-free, particles that are well tolerated with minimal to no adverse effects because they constitute a natural communication pathway for the cells to share information among themselves. An advantage of the present invention is that it poses no concern regarding human leukocyte antigen (HLA) or major histocompatibility complex (MHC) incompatibility. The exosomes used in the invention can come from a universal donor, such as the universal donor property of Exosome Therapeutics, Inc.

### SUMMARY OF THE INVENTION

The present invention overcomes these and other deficiencies of the prior art by providing cGMP autologous and/or universal donor exosomes loaded with cGMP grade DNA plasmids and any of its forms with or without RNA plasmids to confer a long-lasting effect of self-producing neutralizing antibodies against any or both of the isoforms of SGLT receptors for the treatment of T1DM, T2DM, ASCVD, NASH, NAFLD, and A1ATD.

In one embodiment, the invention is related to an exosome comprising: bioengineered RNAi; plasmid DNA; DNA correction; and modification materials. In another embodiment, the invention is related to an exosome comprising: proteins; small molecules; and peptides. In another embodiment, the invention is related to a method of using an exosome comprising the step of; loading the at least one or more cargos into an exosome, wherein the exosome is of cGMP grade quality. In specific embodiments, the loading of the at least one or more cargos into an exosome is simultaneous in order to have a dual effect. In other embodiments, loading at least one or more cargos into an exosome having cGMP grade quality is sequential.

One aspect of the invention is related to therapeutic agents consisting of cGMP grade autologous exosomes loaded with cGMP grade materials that inhibit or neutralize SGLTs. In one embodiment, an exosome comprises: small interference RNA (siRNA); GalNAc (N-Acetylgalactosamine); a plasmid DNA, or a combination thereof. In another embodiment, an exosome comprises: another RNAi technology; GalNAc; a plasmid DNA; or a combination thereof. In certain embodiments, the plasmid is used alone as monotherapy in preclinical and clinical trials as well as for human use. In certain embodiments, a cGMP grade autologous exosome comprises cGMP grade materials that transduce cells to correct the ‘Z’ mutation and to express sustainable, consistent levels of normal, ‘M’ AAT. In one embodiment an exosome comprises DNAi and any of its forms with or without DNA plasmids. In certain embodiments an exosome comprises: bioengineered RNAi; plasmid DNA; DNA correction; and modification materials or any combination thereof.

The invention also relates to gene editing. Gene editing nucleases and technology are used as needed for the depletion of active sites of SGLT2 or SGLT1 or both in their full length, and transducing cells to express the ‘M’ allele instead of the mutant ‘Z’ allele of the SERPINA1 gene by correcting Lysine to Glutamine (Lys to Glu342) in the SERPINA1 gene by using clustered regularly interspaced short palindromic repeats (CRISPR)/CAS9, Zinc finger, Transcription activator-like effector nucleases (TALENs), megalonucleases and/or single base editing or editors. This also includes Wingless-related integration site (Wnt) signaling targeting, constructs and engineering for nuclear expressions of SGLT1 and/or SGLT2 pathways or alternative pathways. In other embodiments, gene editing comprises wnt signaling targeting, constructs, and engineering for nuclear expressions of relevant main or alternative pathways along with A1ATD and SERPINA1 gene.

In certain embodiments, the DNA plasmid includes doxycycline, ampicillin, kanamycin, another equivalent agent, or a combination thereof. In certain embodiments, the plasmid is used alone as monotherapy in preclinical and clinical trials as well as for human use. In one embodiment, a method of using exosomes comprises the step of implementing ex vivo cell engineering using exosomes to transduce genetic cargo. In another embodiment, a method of using exosomes comprises the step of implementing ex vivo cell engineering using exosomes to integrate genetic cargo into the host genome. In another embodiment, the DNA plasmid undergoes ex vivo or in vivo cell bioengineering of self-producing monoclonal neutralizing antibodies against the ‘Z’ mutant isoforms of AAT inhibiting their cytotoxic properties in hepatocytes and other cells and tissues.

In certain embodiments, the exosome comprises siRNA and at least one DNA construct. The at least one construct is produced by gene editing nucleases technology, insertional mutagenesis, overexpression of target genes, or a combination thereof. In certain embodiments, the exosome comprises siRNA or other RNAi technology.

In certain embodiments, an exosome comprises a retrovirus, lentivirus, AAV, or a combination thereof expressing corrected AAT. In certain embodiments, the lentivirus and/or retrovirus vector comprises a long-term repeat (LTR). In multiple embodiments, the lentivirus and/or retrovirus vector comprises a promoter, wherein the promoter is cytomegalovirus (CMV) or a tissue specific promoter (e.g., lung, liver, etc.), self-inactivating sequence (SIN), vesicular stomatitis virus-G (VSV-G), or a combination thereof. In other embodiments, an exosome comprises a vector expressing a corrected sequence of AAT. In other embodiments, an AAV vector (AAV1-10) comprising an expression sequence for SERPINA1 (M) correcting the production of AAT.

The invention also relates to insertional mutagenesis to correct SERPINA1 point mutation glu342 to lys for the translation of the Z isoform of AAT. In specific embodiments, insertional mutagenesis corrects the point mutation in one or both alleles of a patient. In certain embodiments, the constructs are deployed directly to animals or humans, or a combination thereof. In other embodiments, the constructs undergo ex vivo bioengineering of particular cell lineages.

In various embodiments, a method of using the exosomes to deplete or neutralize SGLT1 and/or SGLT2, a method of using the exosomes include administering an exosomal treatment to a subject or a patient using one selected from the group: intravenous (I.V.), intra-arterial (I.A.), intra-tecal (LT), intra-ventricular (I.Ve), subcutaneous (S.C.), subdermal (S.D)., oral, rectal, intra-peritoneal (LP), transdermal, intraosseous injection/infusion, etc.

In certain embodiments, the exosomes are used to treat ASCVD including Coronary arterial disease (CAD), Peripheral Vascular Disease (PVD), Peripheral Arterial Disease (PAD), ischemic and CHF with preserved or reduced ejection fraction, Stroke, Acute Kidney Failure, Endothelial Dysfunction, Mytochondrial Dysfunction, Oxidative Stress, etc.

In certain embodiments, the exosomes are used to treat Diabetes Mellitus including T1DM, T2DM, or any of their complications such as diabetic foot, diabetic retinopathy, peripheral diabetic neuropathy, diabetic kidney disease, etc. It also includes insulin resistance, pre-diabetes and gestational diabetes.

In certain embodiments, the exosomes are used to treat NAFLD and NASH, and Non-Alcoholic Cirrhosis for primary and/or secondary prevention of its complications such as liver cirrhosis, liver failure, liver cancer, cardiovascular disease (CVD), stroke, and CAD. In certain embodiments, the exosomes comprise cGMP grade therapeutics to treats NAFLD and NASH and its complications including, but not limited to, plasmid DNAs to overexpress glucagon-like peptide-1 (GLP-1)/glucose-dependent insulinotropic polypeptide (GIP)/glucagon or agonists, fatty acid synthase inhibitors (FAS/FASN), uncoupling proteins-1 and 2 (UCP-1/UCP-2), insulin human recombinant (hrInsulin), insulin growth factor (IGF-1), fibroblast growth factors 19 and 21 (FGF19/21), diglyceride acyltransferase or O-acyltransferase (DGAT2), or a combination thereof. In certain embodiments, the exosome comprises cGMP grade therapeutics including, but not limited to, DNA plasmids designed to self-produce monoclonal neutralizing antibodies against SGLT sites, FAS/FASN, thyroid receptor-b (TR-b)/ligands, UCP-1/UCP-2, C-C chemokine receptor types 2 and 5 (CCR2/CCR5), reactive oxygen species (ROS), alpha synuclein (SNCA), hrInsulin/IGF-1, FGF19/21, DGAT2, or a combination thereof.

In certain embodiments, the exosomes are used to treat A1ATD as well as secondary prevention of its complications such as chronic obstructive pulmonary disease (COPD), liver and respiratory problems, and liver cirrhosis related to the deficiency of A1ATD.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 illustrates a method for producing autologous exosomes from a body fluid according to an embodiment of the invention.

FIG. 2 illustrates a method for producing autologous exosomes according to another embodiment of the invention.

FIG. 3 illustrates a method for producing allogenic exosomes from a cell culture according to an embodiment of the invention.

FIG. 4 illustrates a method for producing allogenic exosomes from a body fluid according to another embodiment of the invention.

FIG. 5 illustrates a table of parameters for exosome isolation and purification according to multiple embodiments of the invention.

FIG. 6 illustrates a schematic of siRNA used in combination with GalNAc construct to interact with SGLT2 receptors.

FIG. 7 illustrates a DNA plasmid used to drive overexpression of GLP-1/GIP/Glucagon that is loaded into autologous or universal exosomes.

FIG. 8 illustrates a DNA plasmid used to drive overexpression of FAS/FASN that is loaded into autologous or universal exosomes.

FIG. 9 illustrates self-production of monoclonal neutralizing antibodies against FAS/FASN using a DNA plasmid loaded into autologous or universal exosomes.

FIG. 10 illustrates a DNA plasmid used to drive overexpression of thyroid receptor b (TR-b)/ligands that is loaded into autologous or universal exosomes.

FIG. 11 illustrates self-production of monoclonal neutralizing antibodies against TR-b/agonists using a DNA plasmid loaded into autologous or universal exosomes.

FIG. 12 illustrates a DNA plasmid used to drive overexpression of UCP-1/UCP-2 that is loaded into autologous or universal exosomes.

FIG. 13 illustrates self-production of monoclonal neutralizing antibodies against UCP-1/UCP-2 using a DNA plasmid loaded into autologous or universal exosomes.

FIG. 14 illustrates self-production of monoclonal neutralizing antibodies against CCR2/CCR5/SNCA/ROS using a DNA plasmid loaded into autologous or universal exosomes.

FIG. 15 illustrates a DNA plasmid used to drive overexpression of hrInsulin/IGF-1 that is loaded into autologous or universal exosomes.

FIG. 16 illustrates self-production of monoclonal neutralizing antibodies against hrInsulin/IGF-1 using a DNA plasmid loaded into autologous or universal exosomes.

FIG. 17 illustrates a DNA plasmid used to drive overexpression of FGF19/21 that is loaded into autologous or universal exosomes.

FIG. 18 illustrates self-production of monoclonal neutralizing antibodies against FGF19/21 using a DNA plasmid loaded into autologous or universal exosomes.

FIG. 19 illustrates a DNA plasmid used to drive overexpression of DGAT2 that is loaded into autologous or universal exosomes.

FIG. 20 illustrates self-production of monoclonal neutralizing antibodies against DGAT2 using a DNA plasmid loaded into autologous or universal exosomes.

FIG. 21 illustrates an exosome loaded with siRNA or any other construct mentioned above alone or in combination according to multiple embodiments of the invention.

FIG. 22 illustrates an exosome loaded with combination therapies, siRNA, or any other construct according to multiple embodiments of the invention.

FIG. 23 illustrates a cGMP-grade exosome comprising a cargo comprising a nuclease base editor according to an embodiment of the invention.

FIG. 24 illustrates DNA libraries for SGLT2 or SLC5A2 in humans.

FIG. 25 illustrates DNA libraries for SGLT1 or SLC5A1 in humans.

FIG. 26 illustrates a doxycycline-inducible plasmid DNA.

FIG. 27 illustrates a plasmid DNA.

FIG. 28 illustrates an exosome loaded with a plasmid DNA construct.

FIG. 29 illustrates lentivirus and/or retrovirus driven expression of SERPINA1 (M) according to multiple embodiments of the invention.

FIG. 30 illustrates an exosome loaded with cargo according to multiple embodiments of the invention.

FIG. 31 illustrates an AAV vector (AAV1-10) including the expression sequence for SERPINA1 (M).

FIG. 32 illustrates an exosome loaded with cargo according to multiple embodiments of the invention.

FIG. 33 illustrates insertional mutagenesis according to an embodiments of the invention.

FIG. 34 illustrates gene editing for correcting Lysine to Glutamine in the SERPINA1 gene.

FIG. 35 illustrates siRNA to deplete expression of Z isoform (mutant) of the SERPINA1 gene, according to multiple embodiments of the invention.

FIG. 36 illustrates multiple therapeutic modalities used as monotherapy or combination therapies loaded into exosomes according to multiple embodiments of the invention.

FIG. 37 illustrates SERPINA1 gene (available at https://www.ncbi.nlm.nih.gov/gene/5265).

FIG. 38 illustrates an exosome comprising at least one therapeutic molecule according to multiple embodiments of the invention.

### DETAILED DESCRIPTION OF PREFERRED EMBODIMENTS

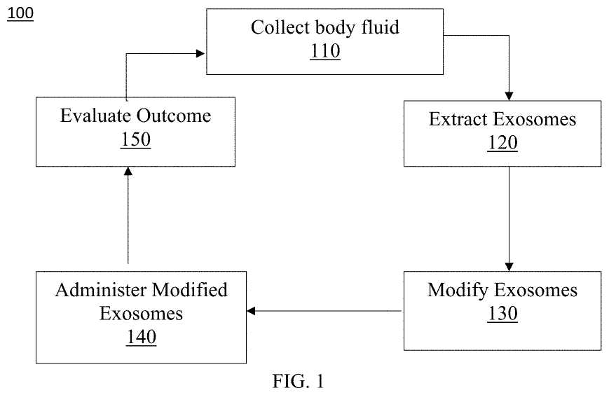

Preferred embodiments of the present invention and their advantages may be understood by referring to FIGS. 1-38. The described features, structures, or characteristics of the invention may be combined in any suitable manner in one or more embodiments. It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Moreover, the described features, structures, or characteristics of the invention may be combined in any suitable manner in one or more embodiments. It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents. Reference will now be made in detail to the preferred embodiments of the invention.

The term “exosome” as used herein refers to any extracellular vesicle derived from any body fluid (e.g., blood) from a human or an animal, any extracellular vesicle derived from human or animal cell lines, cell cultures, and primary cultures not limited to autologous exosomes, universal donor exosomes, allogenic exosomes, and modified exosomes. In certain examples, the exosome is made to meet pharmaceutical and cGMP.

The term “cargo” as used herein refers to any type of molecule or any type of RNA (microRNA, mRNA, tRNA, rRNA, siRNA, RNAi, regulating RNA, gRNA, long interference RNA, non-coding and coding RNA); any type of DNA (DNA fragments, DNA plasmids, iDNA); including any type of nucleic acid including antisense oligonucleotides (ASO); any genetic material; any genetic construct; any nucleic acid construct; any plasmid or vector; any protein including recombinant endogenous protein, enzyme, antibody, wnt signaling proteins; any lipid; any therapeutic molecule or diagnostic molecule; any cellular component; chimeric antigen receptor-T cell (CAR-T cell) transduced without using retroviruses; any virus including retrovirus, adenoviruses (AdV), AAV of any variety and strain, and DNA viruses; any gene editing technology including CRISPR, CRISPR/CAS9 system, any endonucleases for base editing, a Zinc finger, a single base editor, TALENs, any meganuclease; any small molecule; any peptide; any synthetic molecular conjugate; or a combination thereof loaded into an exosome. Typically, such cargo is naturally not present in the exosome. In certain examples, the cargo is made to meet pharmaceutical and cGMP standards.

In one embodiment, the cargo could include a promoter. The term “promoter” as used herein refers to any DNA sequence that promotes the transcription of a gene. A plasmid comprises a tissue-specific promoter. Moreover, the promoter comprises any tissue-specific promoter (e.g., lung, liver, or any other tissue type), a self-inactivating (SIN) sequence, vesicular stomatitis virus-G protein (VSV-G), or a combination thereof. The advantage of using a tissue-specific promoter is to better target a desired tissue in which to transcribe RNA and subsequently encode a protein.

The term “fluid” as used herein refers to any type of body fluid produced by a human or an animal including but not limited to blood, cerebral spinal fluid, urine, saliva, and any biological secretions, etc.

FIGS. 1-4 illustrate methods of producing exosomes and cargo and methods for exosome loading. Such improved methods and techniques would be appreciated by one of ordinary skill in the art, especially those for increasing yield of purified exosomes and efficient loading of exosome cargo for use in preclinical and clinical studies. The methods of loading the genetic material (e.g., constructs of DNA or RNA, or any type of nucleic acids) directly into exosomes are transformation, transfection and microinjection. In one embodiment, exosomes are extracted, isolated and purified from peripheral blood mononuclear cells (PBMC) circulating in peripheral blood. In such an embodiment, PBMCs are harvested from a patient or a universal donor. PBMCs are isolated and expanded in vitro using closed systems for cell culture. In another embodiment, open systems may be used depending on available resources. PBMCs produce and secrete exosomes into the media of a cell culture. The media can be filtered and exosomes can be sorted by specific parameters and purified to improve exosome quality.

The present extracellular vesicular compositions may be used to treat any of the following diseases including, but not limited to: 1. Cancer and oncological disorders including carcinogenesis, malignancies, tumors, metastasis, nodules of any variety (endodermal, mesodermal or ectodermal origin and due to spontaneous mutations or human papillomavirus or other viral infections); 2. Infectious diseases including human immunodeficiency virus and Ebola viral infections; 3. CVD including CAD, peripheral vascular disease, peripheral arterial disease, CHF (ischemic and non-ischemic), stroke, acute kidney failure, endothelial dysfunction, mitochondrial dysfunction, oxidative stress, ASCVD, etc.; 4. Diabetes mellitus including T1DM and T2DM and any of related complications such as diabetic foot, diabetic retinopathy, peripheral diabetic neuropathy, diabetic kidney disease, insulin resistance, pre-diabetes, gestational diabetes, etc.; 5. Non-alcoholic liver disease, NAFLD, NASH, non-alcoholic cirrhosis for primary and secondary prevention; 6. Obesity, overweightness, obesity type-1, type-2, and type 3, morbid obesity, and bariatric surgery; 7. Rare diseases; 8. Gastro-intestinal diseases including Ulcerative colitis, Crohn's disease, etc.; 9. Musculo-skeletal diseases; 10. A1ATD. Further, the present extracellular vesicular compositions may be used for cell therapeutics, vector and cell engineering, pharmacology and toxicology assay development, and similar such processes.

The present invention relates to using modified exosomes associated with cargo for treating Diabetes Mellitus including T1DM and T2DM and any of related complications such as diabetic foot, diabetic retinopathy, peripheral diabetic neuropathy, diabetic kidney disease, insulin resistance, pre-diabetes, gestational diabetes, etc. The present invention further relates to using modified exosomes associated with cargo for treating Non-alcoholic liver disease, NAFLD, NASH, non-alcoholic cirrhosis for primary and secondary prevention. The present invention further relates to using modified exosomes associated with cargo for treating ASCVD and CHF. The present invention further relates to using modified exosomes associated with cargo for treating A1ATD.

FIG. 1 illustrates a method for producing autologous exosomes from a body fluid according to an embodiment of the invention. Although the method 100 is illustrated and described as a sequence of steps, its contemplated that various embodiments of the method 100 may be performed in any order or combination and need not include all of the illustrated steps. The method 100 comprises the step of: collecting body fluid 110 from a subject, extracting exosomes 120 from the body fluid, modifying said exosomes 130, administering modified exosomes 140, and evaluating a health-related outcome 150.

In step 110, body fluid is collected from a subject. The subject may be a human or an animal. The body fluid can be peripheral blood, cerebral spinal fluid, secretions, or any other body fluid in which exosomes can be extracted.

In step 120, exosomes are extracted from the body fluid. The extraction method depends on a number of factors including the type of body fluid extracted. Peripheral blood, for example, contains PBMCs and cellular component layers that can be separated by centrifugation at a medical facility. During the extraction process plasma, cells, and cellular components are kept on dry ice at all times before isolation.

In one embodiment, the body fluid is transported to a laboratory to undergo isolation. In one embodiment, exosome isolation is achieved using a gradient method or a designated isolation kit (i.e., Total Exosome Isolation kit, ThermoFisher). The isolation kit protocol is highly efficient in yielding high amounts of exosomes from a body fluid, a cell culture media, or cell.

The method 100, provides several approaches to further optimize isolation of exosomes and increase exosome yield from a body fluid. In one embodiment, a gradient column separates components of the collected peripheral blood by cell densities. Such cellular densities correspond to exosomes and exosome-related materials. Another embodiment uses an exosome sorting method, where sorting markers or sorting beads are used to isolate exosomes from solution. A further embodiment uses flow cytometry sorting, which uses surface biomarkers present on exosome to identify and sort exosomes and exosome-related materials from cells and cell suspensions. In one embodiment, an exosome can be modified to include a targeting agent on a surface of the exosome.

Specifically, the exosomes can be modified (modified exosomes) to have specific targeting agents such as protein epitopes and similar such targeting agents. In various examples, the modified exosome may have a targeting agent covering an entire surface or a partial surface of the extracellular vesicle. Thin layer chromatography can be used to optimally separate exosomes and exosome-related products according to specific exosome associated surface proteins and lipids. An exosome from peripheral blood, for example, would have exosome-related products such as transferrin receptors (immature exosomes), signaling molecules, and similar cellular components. In another embodiment, ionic separation by drift time can be used to optimize isolating exosomes. For example, mass spectrometry may be used to isolate high yields of exosome and exosome-related products. Ion mobility spectrometry/mass spectrometry may also be performed when physicochemical properties of both the exosome and the cargo need to be defined prior to loading into the exosome. Isolated exosome samples can be purified using column methods in accordance with cGMP protocols and regulatory requirements.

In step 130, the exosomes are modified by incorporating cargos. In one embodiment, the modifications to the exosomes are done ex vivo. The exosomes can be further modified to have specific targeting agents on their surface. Exosomes are assembled or transfected with cargo using a number of methods. In one embodiment, depending on the physicochemical properties of the cargo, the exosomes are assembled or transfected with cargo using liposomes (Lipofectamine 2000, Exofect, or heat shock). In another embodiment, exosomes are assembled or transfected with cargo using retroviruses, AdV, AAV of any variety and strain. In another embodiment, exosomes are assembled or transfected with cargo using DNA viruses, sRNAi, long interference RNA, noncoding RNA, RNAi, RNA vectors. In another embodiment, exosomes are assembled or transfected with cargo using DNA, DNA plasmids, CRISPR, CRISPR/CAS9 and/or any endonucleases for gene editing. In another embodiment, exosomes are assembled or transfected with cargo using gene editing technology, small molecules, antibodies, and proteins including recombinant endogenous proteins. In another embodiment, exosomes are assembled or transfected with cargo using oligonucleotide therapeutics, including ASO, gene targeting technology, and gene correction technology. In another embodiment, exosomes are assembled or transfected with cargo using synthetic/molecular conjugates and physical methods for delivery of gene and cell therapeutics.

In step 130, the method for loading exosomes efficiently and effectively incorporates autologous or exogenous materials (therapeutic compounds above or any endogenous enzyme, protein, lipid, molecule, DNA or RNA of interest). In non-limiting examples, the method for loading an exosome can include the process of: 1) Lipid-lipid affinity, using liposomes of high and low density; 2) Incorporating intracellular affinity proteins and/or molecules into the exosome; 3) Using clathrin-coated vesicles in clathrin-mediated endocytosis methods for incorporation of a therapeutic molecule into an exosome or an exosome-like carrier; and 4) Endocytosis receptors/proteins methodology. In one embodiment the method for loading exosomes includes the methods of exosome membrane dissociation and reconstitution via chemical or electromagnetic gradient changes. In another embodiment, a method is used for large molecules or heavy compounds. In certain examples, the optimization of the method 100 is due to including transmembrane transporters activators when loading the biological materials into the exosomes. After the exosome has been loaded, any potential activator remaining in the exosome will be filtered and purified using column methods in compliance with cGMP and regulatory requirements before undergoing the next processing steps.

Exosomes loaded with cargo are considered mature exosomes and are inspected for cGMP compliance, purity and stability for quality assurance and quality check. Next, mature exosomes that have passed the quality check undergo an expansion process if needed. Next, the mature exosomes are diluted and premix into saline/vehicle (depending on the characteristics of the cargo) for a ready to administer tube/cartridge. Finally, the suspension is frozen and stored or shipped to a site for use in clinical or preclinical studies and to patients for self-injection of approved-clinical grade mature exosomes.

In step 140, the mature exosomes are administered to the subject. The subject, may be the same subject from which the body fluid was collected in step 110. The method of administering the exosomes 140 includes, but is not limited to: I.V., I.A., I.T., I.Ve., subcutaneous, subdermal, oral, rectal, I.P., transdermal, intraosseous injection, intraosseous infusion, or a combination thereof. In one embodiment the mature exosomes are administered in vivo.

In step 150, the outcome of the treatment is evaluated. This evaluation can be done using a variety of methods, which is immediately apparent to one of ordinary skill in the art.

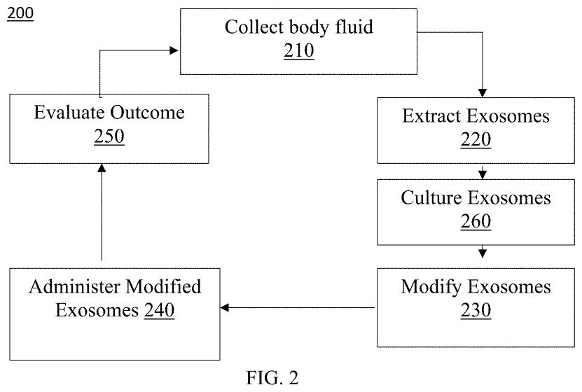

FIG. 2 illustrates a method for producing autologous exosomes from a body fluid according to an embodiment of the invention. Although the method 200 is illustrated and described as a sequence of steps, its contemplated that various embodiments of the method 200 may be performed in any order or combination and need not include all of the illustrated steps. The method 200 comprises the step of: collecting body fluid 210 from a subject, extracting exosomes 220 from the body fluid, culture the exosomes 260, modifying the exosomes 230, administering modified exosome 240, and evaluating the outcome 250.

In step 210, body fluid is collected from a subject. The subject may be a human or an animal. The body fluid can be peripheral blood, cerebral spinal fluid, secretions, or any other body fluid in which exosomes can be extracted.

In step 220, exosomes are extracted from the body fluid using methods as explained above.

In step 260 the exosomes are subjected to a primary culture and expansion. The exosomes will be extracted from primary cultured cells using a gradient or filtration method or a designated expansion kit (i.e., Total Exosome Isolation kit (from cell culture media), ThermoFisher). The cell culture and expansion may be frozen and stored for future exosome isolation procedures/protocols per the methods described above.

In step 230, the exosomes are modified by incorporating cargos. Exosomes are assembled or transfected with cargo using a number of methods as explained above. In one embodiment the step of modifying the exosomes occurs ex vivo.

In step 240 the mature exosomes are administered to a subject using methods as explained above. The step of administering the modified exosomes can occur in vivo or in vitro.

In step 250, the outcome of the treatment is evaluated. This evaluation can be done using a variety of methods, which is immediately apparent to one of ordinary skill in the art.

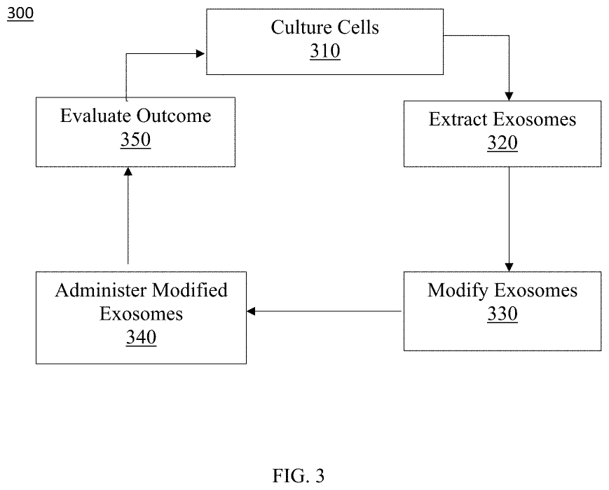

FIG. 3 illustrates a method for producing autologous exosomes from a cell culture according to an embodiment of the invention. Although the method 300 is illustrated and described as a sequence of steps, its contemplated that various embodiments of the method 300 may be performed in any order or combination and need not include all of the illustrated steps. The method 300 comprises the step of: culturing cells 310, extracting exosomes 320 from the cell culture, modifying the exosomes 330, administering modified exosome 340, and evaluating the outcome 350.

In step 310, primary or stable cell lines of human or animal origin are cultured and expanded with standard conditions.

In step 320, exosomes are extracted from the cultured cells.

In step 330, the exosomes are modified by incorporating cargos. Exosomes are assembled or transfected with cargo using a number of methods as explained above.

In step 340 the mature exosomes are administered to a subject using methods as explained above.

In step 350, the outcome of the treatment is evaluated. This evaluation can be done using a variety of methods, which is immediately apparent to one of ordinary skill in the art.

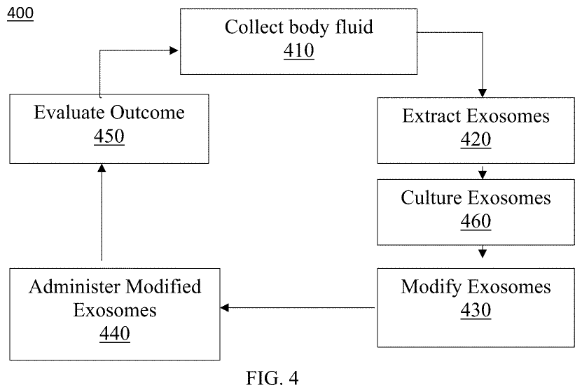

FIG. 4 illustrates a method for producing autologous exosomes from body fluid according to an embodiment of the invention. Although the method 400 is illustrated and described as a sequence of steps, its contemplated that various embodiments of the method 400 may be performed in any order or combination and need not include all of the illustrated steps. The method 400 comprises the step of: collecting body fluid 410, extracting exosomes 420 from the body fluid, culturing the exosomes 460, modifying the exosomes 430, administering modified exosome 440, and evaluating the outcome 450.

In step 410, a body fluid is collected from a universal donor or patient. The subject may be a human or an animal. The body fluid can be peripheral blood, cerebral spinal fluid, secretions, or any other body fluid in which exosomes can be extracted.

In step 420, exosomes are extracted from the body fluid using methods as explained above.

In step 460, the exosomes are cultured. The exosomes are expanded using a primary cell culture from the body fluid of the universal donor or patient using a gradient method or a designated isolation kit (i.e., Total Exosome Isolation kit, ThermoFisher). The isolation kit protocol is highly efficient in yielding high amounts of exosomes from either body fluids or cell culture media or cell. The cell culture and expansion from the universal donor or patient may be frozen and stored for future exosome isolation procedures/protocols per the methods described above.

In step 430, the exosomes are modified by incorporating cargos. Exosomes are assembled or transfected with cargo using a number of methods as explained above.

In step 440 the mature exosomes are administered to a subject using methods as explained above.

In step 450, the outcome of the treatment is evaluated. This evaluation can be done using a variety of methods, which is immediately apparent to one of ordinary skill in the art.

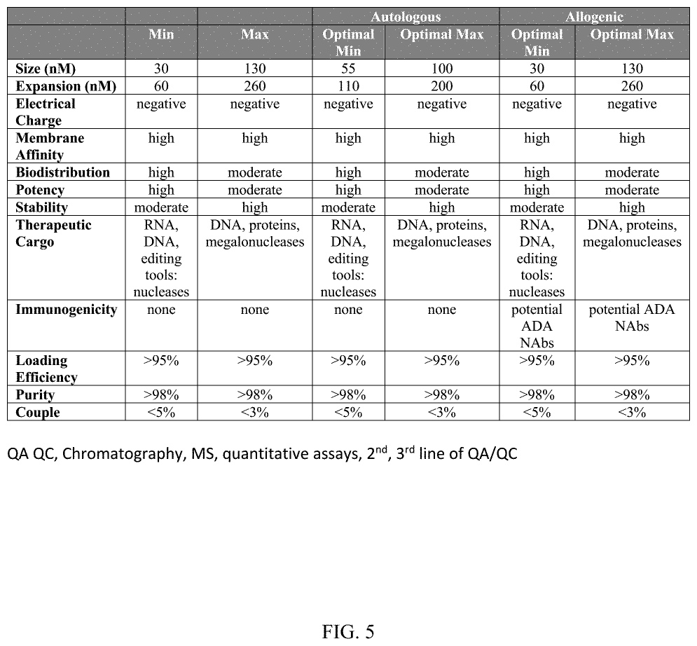

FIG. 5 illustrates the parameters used to sort exosomes according to an embodiment of the invention. In one embodiment, the invention provides autologous exosomes having a vesicle size between about 55 nanometers (nM) and 100 nM. In certain embodiments, allogenic exosomes have a vesicle size between about 30 nM and 130 nM. A vesicle size between 55 nM and 100 nM may be chosen as larger exosomes are less stable. Also, larger exosomes can couple with other exosomes making calculating drug dose, bioavailability, and biodistribution challenging. In some embodiments, the exosomes have the ability to expand to a size between about 60 nM and 260 nM. Such expanded exosomes can encompass large constructs. In some embodiments, the expanded exosomes can encompass more than or equal to about 7 kilo bases (Kb), and accommodate one or more copies of a relatively large viral particle such as an AAV. In one example, an exosome is loaded with at least four AAV particles to improve an exosome safety profile. In some embodiments, either an autologous or allogenic exosome has a negative electrical charge. Both autologous and allogenic exosomes may have a high membrane affinity. In some embodiments, biodistribution may be moderate to high. In a similar embodiment, potency may range from high to moderate while stability may be moderate to high.

In an embodiment, exosomes can comprise a smaller sized cargo comprising RNA, DNA, editing tools (e.g., nucleases), or a combination thereof. In another embodiment, an exosome can comprise a larger cargo comprising DNA, proteins, megalonucleases, or a combination thereof. One advantage of autologous exosomes is that they do no illicit a significant immune response. Allogenic exosomes may illicit anti-drug antibodies (ADA) and neutralizing antibodies (NAb). One embodiment of the present invention enables high efficiency of loading cargo into at least ninety-five percent (95%) of exosomes. Another embodiment can provide a higher purity of exosomes of at least ninety-eight percent (98%).

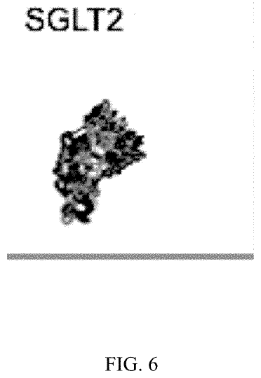

FIG. 6 illustrates an siRNA used in combination with GalNAc construct to interact with SGLT2 receptors. In another embodiment, siRNA in combination with GalNAc construct could be used to interact with SGLT1 receptors. The siRNA and/or siRNA-GalNAc constructs are loaded into autologous or universal exosomes for depletion (short acting and long acting) according to multiple embodiments of the invention. Such exosomes are prepared according to the methods described above in various embodiments. The invention provides an exosome with cargo comprising siRNA, a GalNAc construct, or a combination thereof. In one embodiment, the siRNA and/or siRNA-GalNAc constructs are used to downregulate the SGLTs. In one embodiment, the siRNA and siRNA-GalNAc constructs are modified to enable greater loading efficacy.

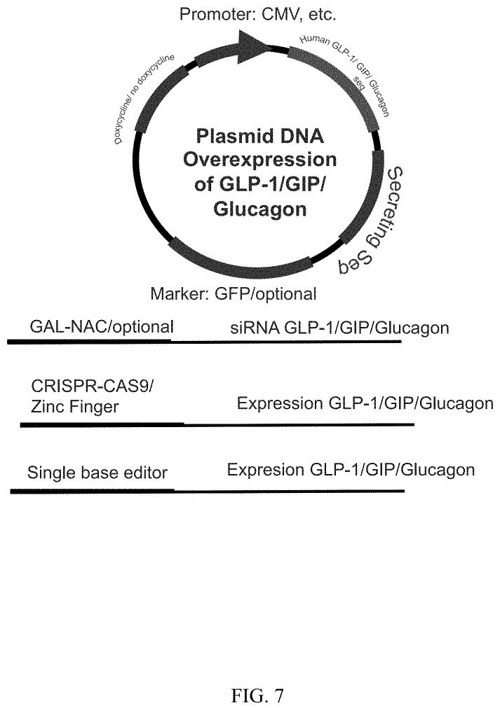

FIG. 7 illustrates a DNA plasmid used to drive overexpression of human GLP-1/GIP/glucagon that is loaded into autologous or universal exosomes. In certain embodiments, the target gene is GLP-1/GIP/glucagon, human FAS/FASN, human TR-b/ligands, human UCP-1/UCP-2, hrInsulin/GF-1, human FGF19/21, human DGAT2, or a combination thereof. In certain embodiments, the DNA plasmid contains a promoter (e.g., CMV, liver specific, etc.). In specific embodiments, the DNA plasmid includes doxycycline, ampicillin, kanamycin, another equivalent agent, or a combination thereof. In specific embodiments, the DNA plasmid contains a secreting sequence. In specific embodiments, the DNA plasmid may contain a marker such as green fluorescent protein (GFP).

In one embodiment, an exosome may include gene editing nucleases and technology as needed for targeting GLP-1/GIP/Glucagon or any other target gene using CRISPR, CRISPR/CAS9, Zinc finger, TALENs, megalonucleases and/or single base editing or editors. This may also include Wnt signaling targeting, wherein engineering for nuclear expressions of GLP-1/GIP/Glucagon, SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

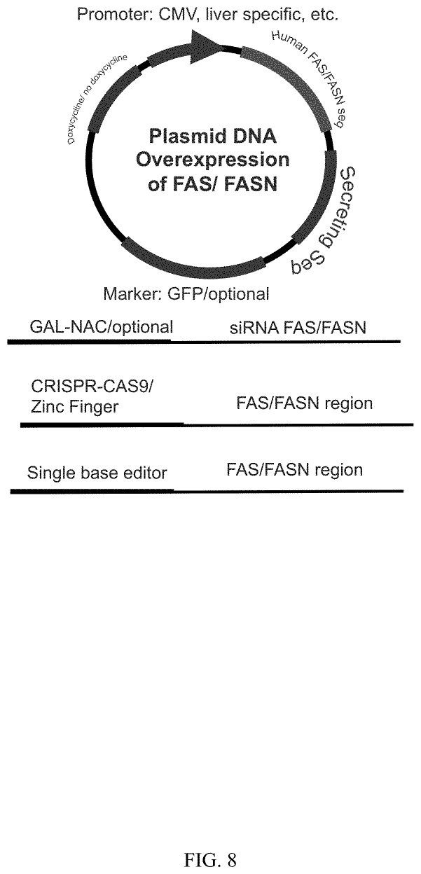

FIG. 8 illustrates a DNA plasmid used to drive overexpression of FAS/FASN that is loaded into autologous or universal exosomes. In one embodiment, the DNA plasmid targets human FAS/FASN overexpression that is loaded into autologous or universal exosomes. In certain embodiments, the target gene is human GLP-1/GIP/glucagon, human TR-b/ligands, human UCP-1/UCP-2, human hrInsulin/GF-1, human FGF19/21, human DGAT2, or a combination thereof. In certain embodiments, the DNA plasmid contains a promoter (e.g., CMV, liver specific, etc.). In specific embodiments, the DNA plasmid includes doxycycline, ampicillin, kanamycin, another equivalent agent, or a combination thereof. In specific embodiments, the DNA plasmid contains a secreting sequence. In specific embodiments, the DNA plasmid may contain a marker such as green fluorescent protein (GFP).

In one embodiment, an exosome may include gene editing nucleases and technology as needed for targeting FAS/FASN or any other target gene using CRISPR, CRISPR/CAS9, Zinc finger, TALENs, megalonucleases and/or single base editing or editors. This may also include Wnt signaling targeting, wherein engineering for nuclear expressions of FAS/FASN, SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

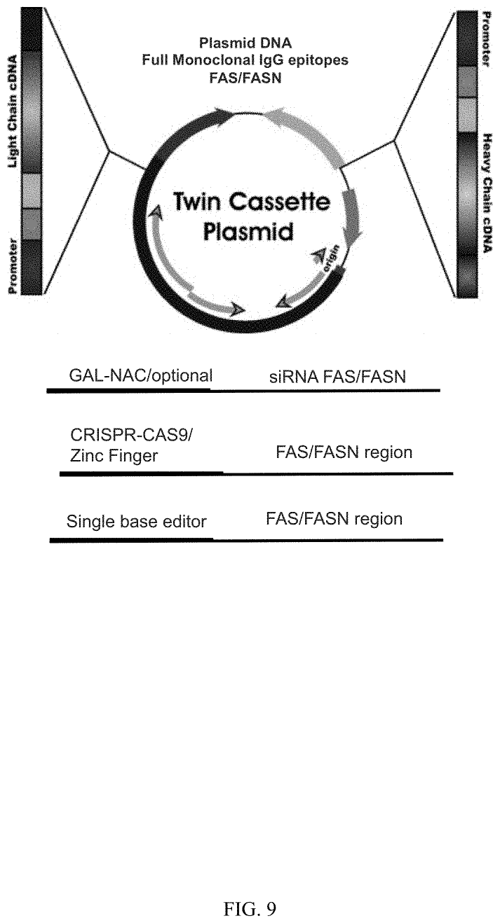

FIG. 9 illustrates self-production of monoclonal neutralizing antibodies against FAS/FASN using a DNA plasmid loaded into autologous or universal exosomes. In one embodiment, the target genes are FAS/FASN using a DNA plasmid for the self-production of monoclonal neutralizing antibodies loaded into autologous or universal exosomes. In certain embodiments, the DNA plasmid targets the active sites of the SGLT1 and/or SGLT2 receptors, TR-b/agonists, UCP-1/UCP-2, CCR2 and CCR5, ROS, SNCA, hrInsulin, IGF1, FGF19/21, DGAT2, or O-acyltransferase, or a combination thereof. Such exosomes are prepared according to the methods described above in various embodiments. The DNA plasmid can be used for the ex vivo or in vivo cell bioengineering of the self-producing monoclonal neutralizing antibodies. In specific embodiments, the DNA plasmid includes doxycycline, ampicillin, kanamycin, another equivalent agent, or a combination thereof. In one embodiment, an exosome includes siRNA, GalNAc construct, plasmid DNA, or a combination thereof. In another embodiment, an exosome can include another RNAi technology, GalNAc construct, a plasmid DNA, or a combination thereof. In certain embodiments, the plasmid is used alone as monotherapy in preclinical and clinical trials as well as for human use. In one embodiment, plasmid DNA is modified to enable greater loading efficacy.

In one embodiment, an exosome may include gene editing nucleases and technology as needed for targeting of FAS/FASN using CRISPR, CRISPR/CAS9, Zinc finger, TALENs, megalonucleases and/or single base editing or editors. This may also include Wnt signaling targeting, wherein engineering for nuclear expressions of SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

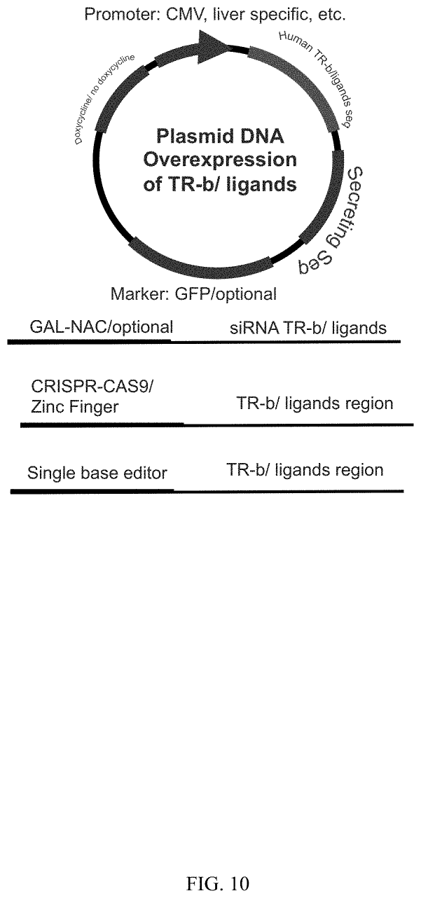

FIG. 10 illustrates a DNA plasmid used to drive overexpression of thyroid receptor b (TR-b)/ligands that is loaded into autologous or universal exosomes. In one embodiment, the DNA plasmid targets TR-b/ligands that is loaded into autologous or universal exosomes. In certain embodiments, the target gene is human GLP-1/GIP/glucagon, human FAS/FASN, human UCP-1/UCP-2, human hrInsulin/GF-1, human FGF19/21, human DGAT2, or a combination thereof. In certain embodiments, the DNA plasmid contains a promoter (e.g., CMV, liver specific, etc.). In specific embodiments, the DNA plasmid includes doxycycline, ampicillin, kanamycin, another equivalent agent, or a combination thereof. In specific embodiments, the DNA plasmid contains a secreting sequence. In specific embodiments, the DNA plasmid may contain a marker such as green fluorescent protein (GFP).

In one embodiment, an exosome may include gene editing nucleases and technology as needed for targeting TR-b/ligands or any other target gene using CRISPR, CRISPR/CAS9, Zinc finger, TALENs, megalonucleases and/or single base editing or editors. This may also include Wnt signaling targeting, wherein engineering for nuclear expressions of TR-b/ligands, SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

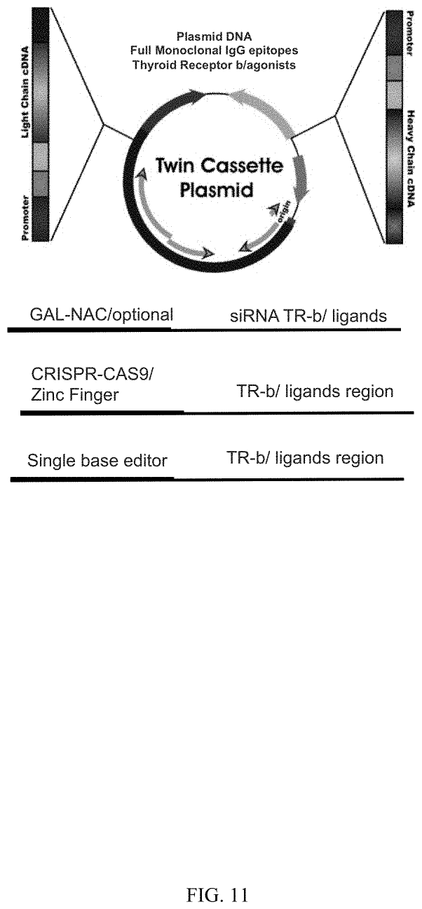

FIG. 11 illustrates self-production of monoclonal neutralizing antibodies against TR-b/agonists using a DNA plasmid loaded into autologous or universal exosomes. In one embodiment, the target is TR-b/agonists using a DNA plasmid for the self-production of monoclonal neutralizing antibodies loaded into autologous or universal exosomes. In certain embodiments, the DNA plasmid targets the active sites of the SGLT1 and/or SGLT2 receptors, FAS/FASN, UCP-1/UCP-2, CCR2 and CCR5, ROS, SNCA, hrInsulin, IGF1, FGF19/21, DGAT2, or O-acyltransferase, or a combination thereof. Such exosomes are prepared according to the methods described above in various embodiments. The DNA plasmid can be used for the ex vivo or in vivo cell bioengineering of the self-producing monoclonal neutralizing antibodies. In specific embodiments, the DNA plasmid includes doxycycline, ampicillin, kanamycin, another equivalent agent, or a combination thereof. In one embodiment, an exosome includes siRNA, GalNAc construct, plasmid DNA, or a combination thereof. In another embodiment, an exosome can include another RNAi technology, GalNAc construct, a plasmid DNA, or a combination thereof. In certain embodiments, the plasmid is used alone as monotherapy in preclinical and clinical trials as well as for human use. In one embodiment, plasmid DNA is modified to enable greater loading efficacy.

In one embodiment, an exosome may include gene editing nucleases and technology as needed for the depletion of TR-b/agonists using CRISPR, CRISPR/CAS9, Zinc finger, TALENs, megalonucleases and/or single base editing or editors. This may also include Wnt signaling targeting, wherein engineering for nuclear expressions of TR-b/agonists, SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

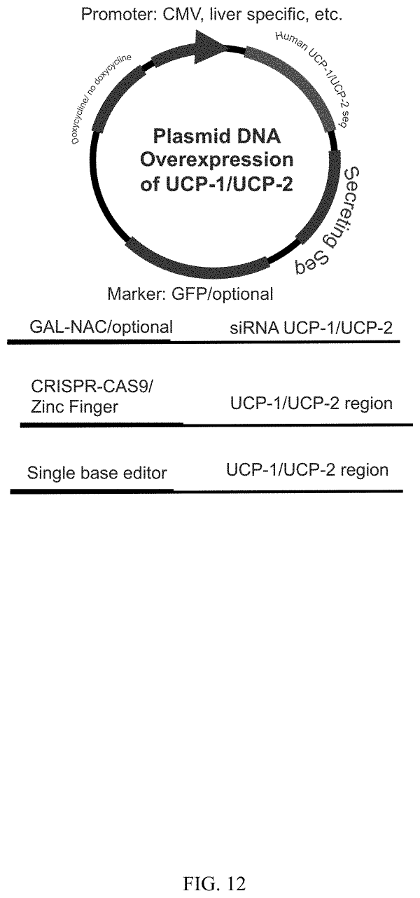

FIG. 12 illustrates a DNA plasmid used to drive overexpression of UCP-1/UCP-2 that is loaded into autologous or universal exosomes. In one embodiment, the DNA plasmid targets UCP-1/UCP-2 that is loaded into autologous or universal exosomes. In certain embodiments, the target gene is human GLP-1/GIP/glucagon, human FAS/FASN, human TR-b/ligands, human hrInsulin/GF-1, human FGF19/21, human DGAT2, or a combination thereof. In certain embodiments, the DNA plasmid contains a promoter (e.g., CMV, liver specific, etc.). In specific embodiments, the DNA plasmid includes doxycycline, ampicillin, kanamycin, another equivalent agent, or a combination thereof. In specific embodiments, the DNA plasmid contains a secreting sequence. In specific embodiments, the DNA plasmid may contain a marker such as green fluorescent protein (GFP).

In one embodiment, an exosome may include gene editing nucleases and technology as needed for the targeting UCP-1/UCP-2 or any other target gene using CRISPR, CRISPR/CAS9, Zinc finger, TALENs, megalonucleases and/or single base editing or editors. This may also include Wnt signaling targeting, wherein engineering for nuclear expressions of UCP-1/UCP-2, SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

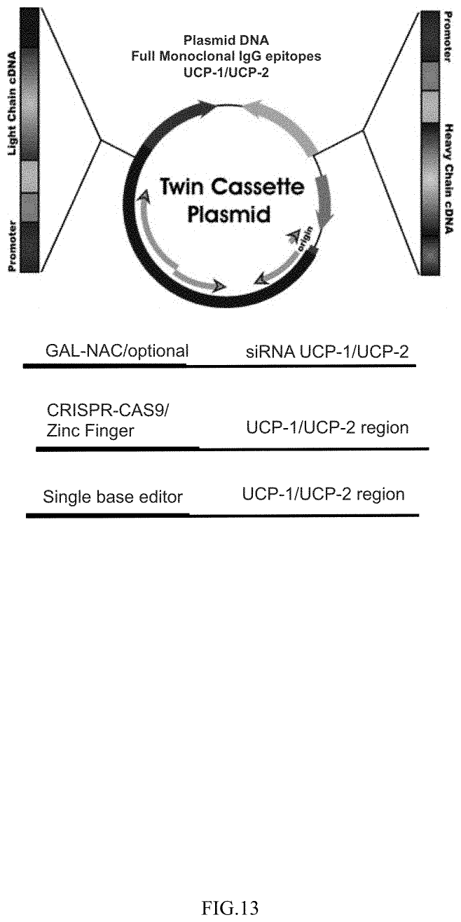

FIG. 13 illustrates self-production of monoclonal neutralizing antibodies against UCP-1/UCP-2 using a DNA plasmid loaded into autologous or universal exosomes. In one embodiment, the DNA plasmid targets UCP-1/UCP-2 using a DNA plasmid for the self-production of monoclonal neutralizing antibodies loaded into autologous or universal exosomes. In certain embodiments, the target genes are FAS/FASN, TR-b/agonists, CCR2 and CCR5, ROS, SNCA, hrInsulin, IGF1, FGF19/21, DGAT2 or O-acyltransferase, or a combination thereof. Such exosomes are prepared according to the methods described above in various embodiments. The DNA plasmid can be used for the ex vivo or in vivo cell bioengineering of the self-producing monoclonal neutralizing antibodies. In specific embodiments, the DNA plasmid includes doxycycline, ampicillin, kanamycin, another equivalent agent, or a combination thereof. In one embodiment, an exosome includes siRNA, GalNAc construct, a plasmid DNA, or a combination thereof. In another embodiment, an exosome can include another RNAi technology, GalNAc construct, a plasmid DNA, or a combination thereof. In certain embodiments, the plasmid is used alone as monotherapy in preclinical and clinical trials as well as for human use. In one embodiment, plasmid DNA is modified to enable greater loading efficacy.

In one embodiment, an exosome may include gene editing nucleases and technology as needed for targeting UCP-1/UCP-2 or any other relevant gene using CRISPR, CRISPR/CAS9, Zinc finger, TALENs, megalonucleases and/or single base editing or editors. This may also include Wnt signaling targeting, wherein engineering for nuclear expressions of UCP-1/UCP-2, SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

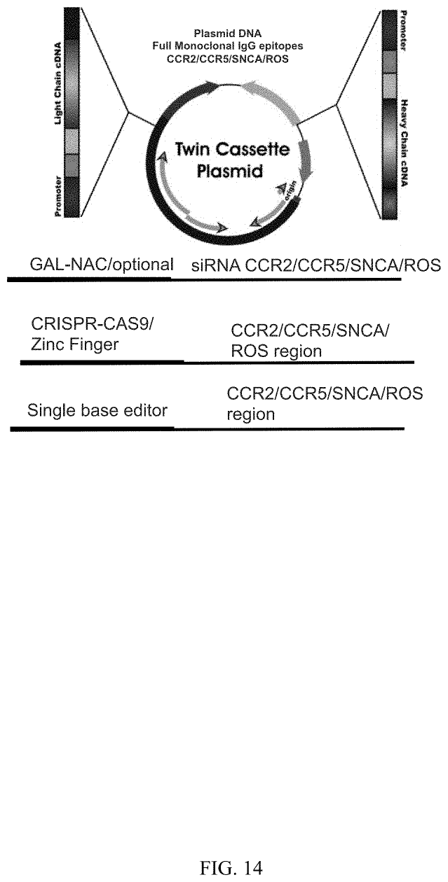

FIG. 14 illustrates self-production of monoclonal neutralizing antibodies against CCR2/CCR5/SNCA/ROS using a DNA plasmid loaded into autologous or universal exosomes. In one embodiment, the DNA plasmid targets CCR2/CCR5/SNCA/ROS using a DNA plasmid for the self-production of monoclonal neutralizing antibodies loaded into autologous or universal exosomes. In certain embodiments, the target genes are FAS/FASN, TR-b/agonists, UCP-1/UCP-2, hrInsulin, IGF1, FGF19/21, DGAT2 or O-acyltransferase, or a combination thereof. Such exosomes are prepared according to the methods described above in various embodiments. The DNA plasmid can be used for the ex vivo or in vivo cell bioengineering of the self-producing monoclonal neutralizing antibodies. In specific embodiments, the DNA plasmid includes doxycycline, ampicillin, kanamycin, another equivalent agent, or a combination thereof. In one embodiment, an exosome includes siRNA, GalNAc construct, a plasmid DNA, or a combination thereof. In another embodiment, an exosome can include another RNAi technology, GalNAc construct, a plasmid DNA, or a combination thereof. In certain embodiments, the plasmid is used alone as monotherapy in preclinical and clinical trials as well as for human use. In one embodiment, plasmid DNA is modified to enable greater loading efficacy.

In one embodiment, an exosome may include gene editing nucleases and technology as needed for targeting CCR2/CCR5/SNCA/ROS or any other relevant gene using CRISPR, CRISPR/CAS9, Zinc finger, TALENs, megalonucleases and/or single base editing or editors. This may also include Wnt signaling targeting, wherein engineering for nuclear expressions of CCR2/CCR5/SNCA/ROS, SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

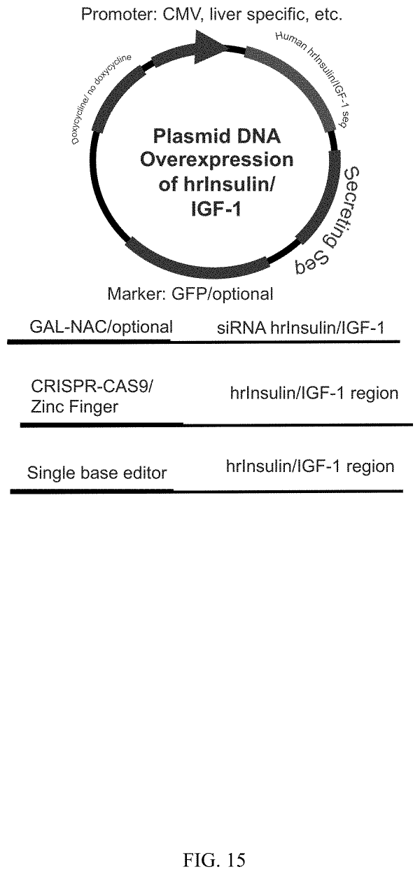

FIG. 15 illustrates a DNA plasmid used to drive overexpression of hrInsulin/IGF-1 that is loaded into autologous or universal exosomes. In one embodiment, the DNA plasmid targets the overexpression of hrInsulin/IGF-1. In certain embodiments, the target gene is human GLP-1/GIP/glucagon, human FAS/FASN, human TR-b/ligands, human UCP-1/UCP-2, human FGF19/21, human DGAT2, or a combination thereof. In certain embodiments, the DNA plasmid contains a promoter (e.g., CMV, liver specific, etc.). In specific embodiments, the DNA plasmid includes doxycycline, ampicillin, kanamycin, another equivalent agent, or a combination thereof. In specific embodiments, the DNA plasmid contains a secreting sequence. In specific embodiments, the DNA plasmid may contain a marker such as green fluorescent protein (GFP).

In one embodiment, an exosome may include gene editing nucleases and technology as needed for targeting hrInulin/IGF-1 or any other target gene using CRISPR, CRISPR/CAS9, Zinc finger, TALENs, megalonucleases and/or single base editing or editors. This may also include Wnt signaling targeting, wherein engineering for nuclear expressions of hrInulin/IGF-1, SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

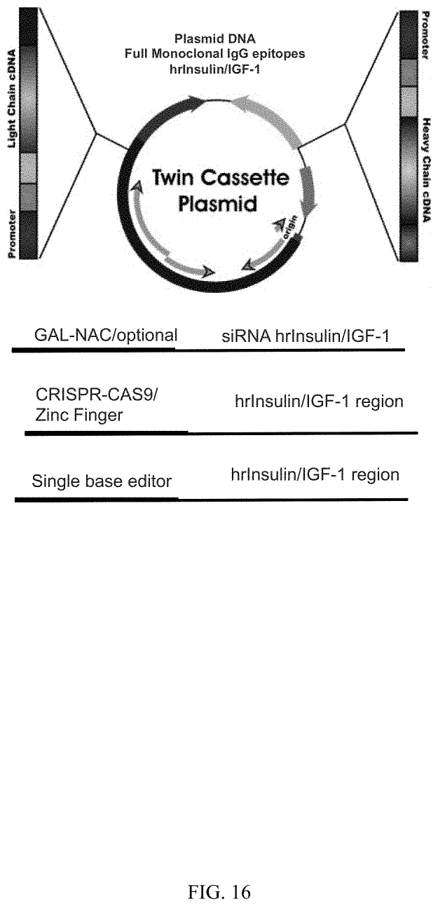

FIG. 16 illustrates self-production of monoclonal neutralizing antibodies against hrInsulin/IGF-1 using a DNA plasmid loaded into autologous or universal exosomes. In one embodiment, the DNA plasmid targets hrInulin/IGF-1 using a DNA plasmid for the self-production of monoclonal neutralizing antibodies loaded into autologous or universal exosomes. In certain embodiments, the target genes are FAS/FASN, TR-b/agonists, UCP-1/UCP-2, CCR2 and CCR5, ROS, SNCA, FGF19/21, DGAT2 or O-acyltransferase, or a combination thereof. Such exosomes are prepared according to the methods described above in various embodiments. The DNA plasmid can be used for the ex vivo or in vivo cell bioengineering of the self-producing monoclonal neutralizing antibodies. In specific embodiments, the DNA plasmid includes doxycycline, ampicillin, kanamycin, another equivalent agent, or a combination thereof. In one embodiment, an exosome includes siRNA, GalNAc construct, a plasmid DNA, or a combination thereof. In another embodiment, an exosome can include another RNAi technology, GalNAc construct, a plasmid DNA, or a combination thereof. In certain embodiments, the plasmid is used alone as monotherapy in preclinical and clinical trials as well as for human use. In one embodiment, plasmid DNA is modified to enable greater loading efficacy.

In one embodiment, an exosome may include gene editing nucleases and technology as needed for targeting hrInulin/IGF-1 using CRISPR, CRISPR/CAS9, Zinc finger, TALENs, megalonucleases and/or single base editing or editors. This may also include Wnt signaling targeting, wherein engineering for nuclear expressions of hrInulin/IGF-1, SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

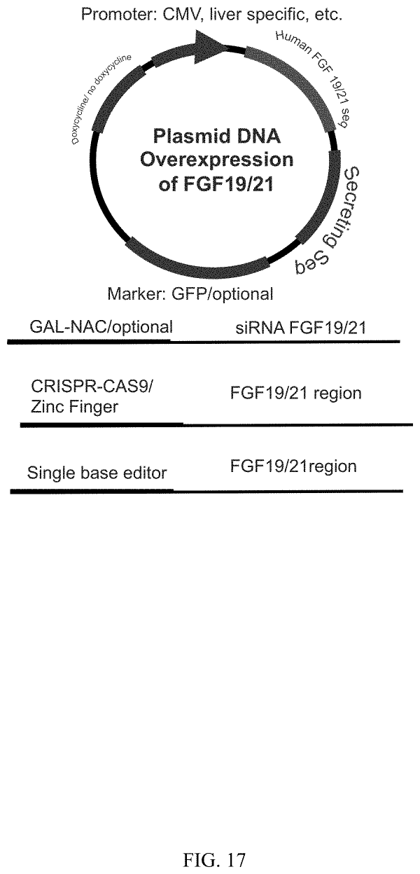

FIG. 17 illustrates a DNA plasmid used to drive overexpression of FGF19/21 that is loaded into autologous or universal exosomes. In one embodiment, the DNA plasmid targets the overexpression of FGF19/21. In certain embodiments, the target gene is human GLP-1/GIP/glucagon, human FAS/FASN, human TR-b/ligands, human UCP-1/UCP-2, human hrInsulin/GF-1, human DGAT2, or a combination thereof. In certain embodiments, the DNA plasmid contains a promoter (e.g., CMV, liver specific, etc.). In specific embodiments, the DNA plasmid includes doxycycline, ampicillin, kanamycin, another equivalent agent, or a combination thereof. In specific embodiments, the DNA plasmid contains a secreting sequence. In specific embodiments, the DNA plasmid may contain a marker such as green fluorescent protein (GFP).

In one embodiment, an exosome may include gene editing nucleases and technology as needed for targeting FGF19/21 or any other target gene using CRISPR, CRISPR/CAS9, Zinc finger, TALENs, megalonucleases and/or single base editing or editors. This may also include Wnt signaling targeting, wherein engineering for nuclear expressions of FGF19/21, SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

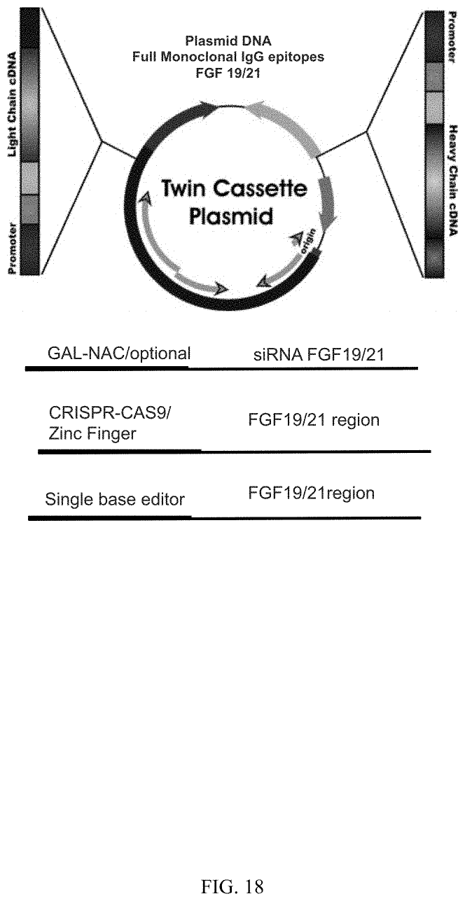

FIG. 18 illustrates self-production of monoclonal neutralizing antibodies against FGF19/21 using a DNA plasmid loaded into autologous or universal exosomes. In one embodiment, the DNA plasmid targets FGF19/21 using a DNA plasmid for the self-production of monoclonal neutralizing antibodies loaded into autologous or universal exosomes. In certain embodiments, the target genes are FAS/FASN, TR-b/agonists, UCP-1/UCP-2, CCR2 and CCR5, ROS, SNCA, hrInsulin, IGF1, DGAT2 or O-acyltransferase, or a combination thereof. Such exosomes are prepared according to the methods described above in various embodiments. The DNA plasmid can be used for the ex vivo or in vivo cell bioengineering of the self-producing monoclonal neutralizing antibodies. In specific embodiments, the DNA plasmid includes doxycycline, ampicillin, kanamycin, another equivalent agent, or a combination thereof. In one embodiment, an exosome includes siRNA, GalNAc construct, a plasmid DNA, or a combination thereof. In another embodiment, an exosome can include another RNAi technology, GalNAc construct, a plasmid DNA, or a combination thereof. In certain embodiments, the plasmid is used alone as monotherapy in preclinical and clinical trials as well as for human use. In one embodiment, plasmid DNA is modified to enable greater loading efficacy.

In one embodiment, an exosome may include gene editing nucleases and technology as needed for targeting FGF19/21 using CRISPR, CRISPR/CAS9, Zinc finger, TALENs, megalonucleases and/or single base editing or editors. This may also include Wnt signaling targeting, wherein engineering for nuclear expressions of FGF19/21, SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

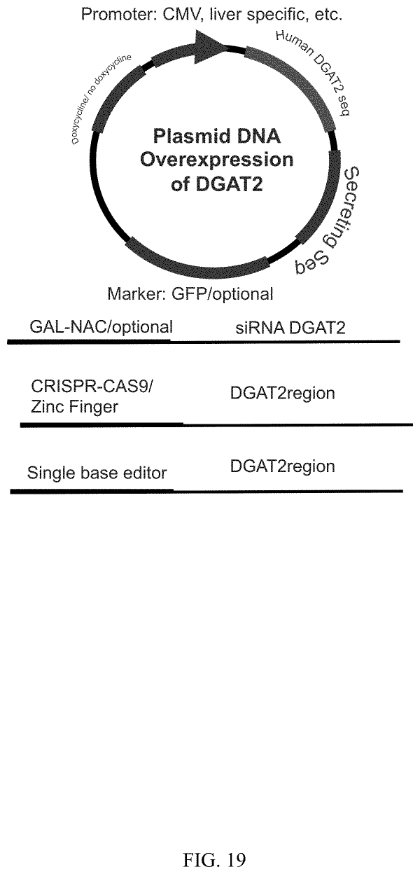

FIG. 19 illustrates a DNA plasmid used to drive overexpression of DGAT2 that is loaded into autologous or universal exosomes. In certain embodiments, the target gene is human GLP-1/GIP/glucagon, human FAS/FASN, human TR-b/ligands, human UCP-1/UCP-2, human hrInsulin/GF-1, human FGF19/21, human DGAT2, or a combination thereof. In certain embodiments, the DNA plasmid contains a promoter (e.g., CMV, liver specific, etc.). In specific embodiments, the DNA plasmid includes doxycycline, ampicillin, kanamycin, another equivalent agent, or a combination thereof. In specific embodiments, the DNA plasmid contains a secreting sequence. In specific embodiments, the DNA plasmid may contain a marker such as green fluorescent protein (GFP).

In one embodiment, an exosome may include gene editing nucleases and technology as needed for targeting DGAT2 or any other target gene using CRISPR, CRISPR/CAS9, Zinc finger, TALENs, megalonucleases and/or single base editing or editors. This may also include Wnt signaling targeting, wherein engineering for nuclear expressions of DGAT2, SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

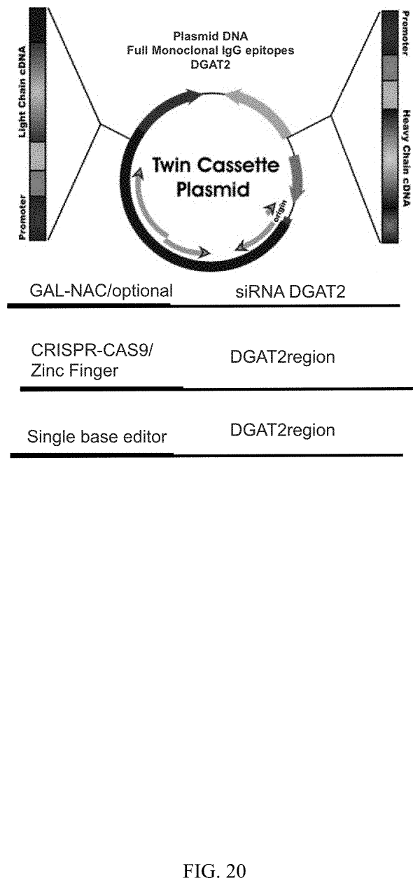

FIG. 20 illustrates self-production of monoclonal neutralizing antibodies against DGAT2 using a DNA plasmid loaded into autologous or universal exosomes. In one embodiment, the DNA plasmid targets DGAT2 using a DNA plasmid for the self-production of monoclonal neutralizing antibodies loaded into autologous or universal exosomes. In another embodiment, the DNA plasmid targets the active sites of the SGLT1 and/or SGLT2 receptors using a DNA plasmid for the self-production of monoclonal neutralizing antibodies loaded into autologous or universal exosomes. In certain embodiments, the target genes are FAS/FASN, TR-b/agonists, UCP-1/UCP-2, CCR2 and CCR5, ROS, SNCA, hrInsulin, IGF1, FGF19/21, or a combination thereof. Such exosomes are prepared according to the methods described above in various embodiments. The DNA plasmid can be used for the ex vivo or in vivo cell bioengineering of the self-producing monoclonal neutralizing antibodies. In specific embodiments, the DNA plasmid includes doxycycline, ampicillin, kanamycin, another equivalent agent, or a combination thereof. In one embodiment, an exosome includes siRNA, GalNAc construct, a plasmid DNA, or a combination thereof. In another embodiment, an exosome can include another RNAi technology, GalNAc construct, a plasmid DNA, or a combination thereof. In certain embodiments, the plasmid is used alone as monotherapy in preclinical and clinical trials as well as for human use. In one embodiment, plasmid DNA is modified to enable greater loading efficacy.

In one embodiment, an exosome may include gene editing nucleases and technology as needed for targeting DGAT2 using CRISPR, CRISPR/CAS9, Zinc finger, TALENs, megalonucleases and/or single base editing or editors. This may also include Wnt signaling targeting, wherein engineering for nuclear expressions of DGAT2, SGLT1 and/or SGLT2 pathways or alternative pathways are apparent to one of ordinary skill in the art.

The resulting exosome with the therapeutic cargo represents a highly efficient, long-lasting, low cost, and straightforward treatment approach. The resulting product provides patients with suitable options, such as chronic therapies for the treatment or co-treatment of T1DM and T2DM, ASCVD, CHF, with preserved or reduced ejection fraction, NAFLD and/or NASH, and A1ATD. Exosomes are effective, virus-free, particles that are well-tolerated with minimal to no adverse effects because they constitute a natural communication pathway for cells to share information among themselves. These novel treatments methods, using exosomes, have no HLA or MHC incompatibility concerns due to the fact that the exosomes are either autologous in nature or are supplied from a universal donor.

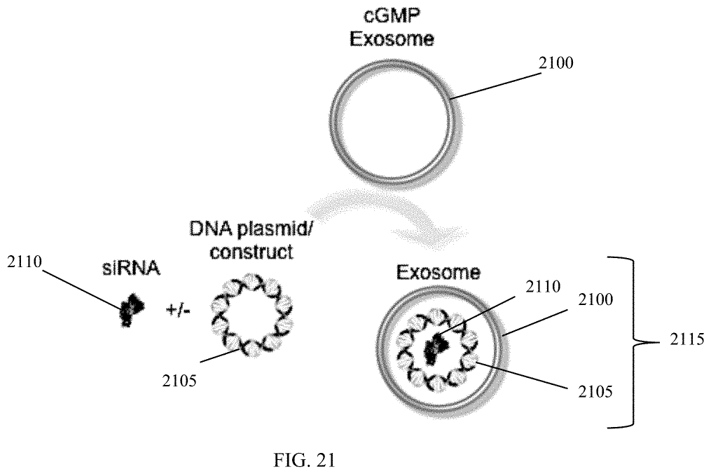

FIG. 21 illustrates a cGMP exosome 2100 being loaded with siRNA 2110 and a construct 2105 according to one embodiment of the invention. The different methods for isolating the cGMP exosome 2100 have been described above. In this embodiment, the cGMP exosome 2100 is loaded with siRNA 2110 and a DNA construct 2105. In one embodiment, the DNA construct 2105 may be plasmid DNA construct. The cGMP exosome 2100 is able to incorporate autologous or exogenous materials (therapeutic compounds above or any endogenous enzyme, protein, lipid, molecule, DNA or RNA of interest) as apparent to a person of ordinary skill in the art. The method for loading the cGMP exosome 2100 can include the process of: 1) Lipid-lipid affinity, using liposomes of high and low density; 2) Incorporating intracellular affinity proteins and/or molecules into the exosome 2100; 3) Using clathrin-coated vesicles in clathrin-mediated endocytosis methods for incorporation of a therapeutic molecule into an exosome or an exosome-like carrier; and 4) Endocytosis receptors/proteins methodology. In one embodiment the method for loading exosomes includes the methods of exosome membrane dissociation and reconstitution via chemical or electromagnetic gradient changes. In this embodiment, when loading the biological materials into the exosomes the cGMP exosome includes transmembrane transporter activators to optimize loading. After the exosome 2100 has been loaded, any potential activator remaining in the exosome 2100 will be filtered and purified using column methods in compliance with cGMP and regulatory requirements before undergoing the next processing steps.

The mature exosome 915 is inspected for cGMP compliance, purity and stability for quality assurance and quality check. Next, the mature exosomes, that have passed the quality check, may undergo an expansion process. Next, the mature exosomes are diluted and premix into saline for a ready to administer tube. Finally, the suspension is frozen and stored or shipped to a site for use in clinical or preclinical studies and to patients for self-injection of approved-clinical grade mature exosomes.

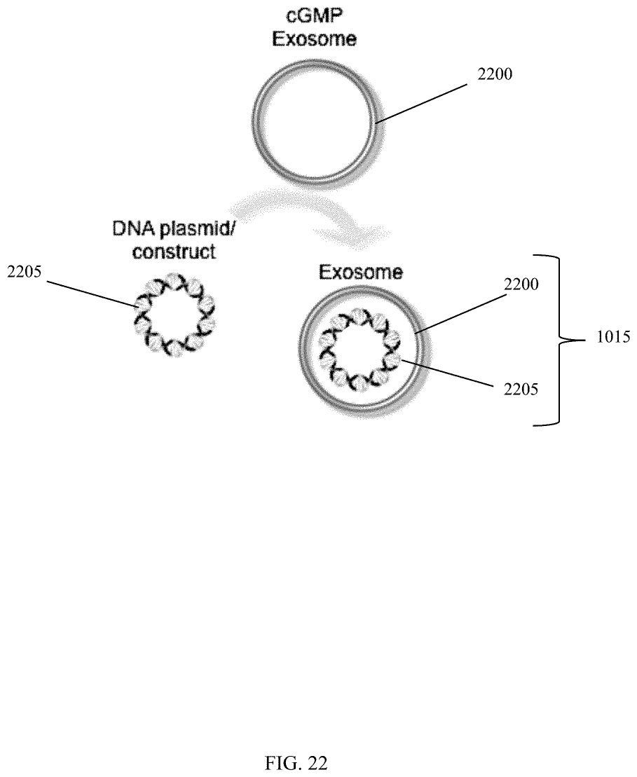

FIG. 22 illustrates a cGMP exosome 2200 loaded with a combination of therapies, specifically, a plasmid DNA construct 2205, according to multiple embodiments of the invention. The different methods for isolating the cGMP exosome 2200 have been described above. The resulting mature exosome 2215 is inspected for cGMP compliance, purity and stability for quality assurance and quality check. As discussed above, the mature exosomes, that have passed the quality check, may undergo an expansion process. Next, the mature exosomes are diluted and premix into saline for a ready to administer tube. Finally, the suspension is frozen and stored or shipped to a site for use in clinical or preclinical studies and to patients for self-injection of approved clinical-grade mature exosomes.

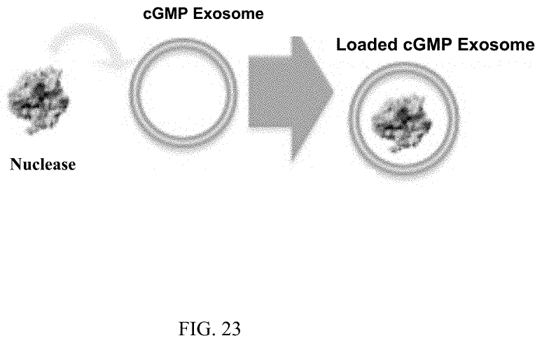

FIG. 23 illustrates a schematic of a cGMP-grade exosome comprising a cargo comprising a nuclease according to an embodiment of the invention. The nuclease functions as a base editor to correct or manipulate the targeted single nucleotide polymorphism (SNP). In one embodiment, in vitro, an exosome-mediated nuclease delivery enables a nuclease to correct the base mutation of cells carrying certain SNPs. In specific examples, a loaded exosome comprising a nuclease base editor is delivered to cells that may be effected by a particular defect. Base editors are the latest generation of gene editing tools with very high precision at targeting single nucleotides within a sequence. Ideally, the loaded exosomes meeting clinical-grade GMP, regulatory chemistry manufacturing and controls (CMC) compliance can be deployed to patients that suffer from T1DM and T2DM, NAFLD, NASH, ASCVD, CHF, A1ATD, and similar such illnesses.

Base editors show very low (0.1%) indel formation (insertion or deletion of bases in the genome), which makes it safe for therapeutic use. A nuclease base editor enables treating certain illnesses by targeting and correcting one or both alleles at a particular DNA sequence. First, a single guide RNA (sgRNA) is designed and added to a nuclease base editor plasmid to increase precision on a target DNA sequence. Second, a protospacer, protospacer adjacent motif (PAM) sequence, and motifs surrounding a particular DNA sequence are included in the target DNA sequence. Inclusion of a protospacer and a PAM sequence enable the CRISPR-CAS9 system to cleave the target DNA sequence. Thirdly, the expression plasmid with sgRNA is cloned. Lastly, the sgRNA and the nuclease base editor are loaded into an exosome. A nuclease base editor corrects one or both alleles at a particular DNA sequence.

FIGS. 21-23 illustrate exosomes loaded with different types of cargo. In an embodiment, any number of cargos discussed may be loaded into a single exosome. Further, the different types of cargo may be loaded into exosomes in any number of combinations. In one embodiment, the exosome may have two or more cargos wherein the two or more cargos may be identical or substantially the same. In another embodiment, an exosome may have two or more cargos wherein each of the two or more cargos are distinct from one another.

In one embodiment, the proportion of loading is 1:1 (exosome:base editor) using loading techniques that have been discussed such as electromagnetism and membrane dissociation technologies. In one embodiment, exosomes having a vesicle size between fifty-five (55) and one hundred (100) nM are selected for cargo loading.

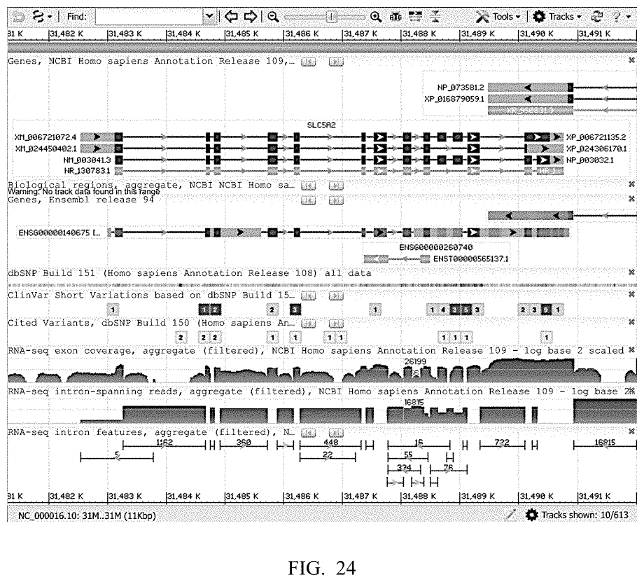

FIG. 24 illustrates the public DNA libraries for SGLT2 receptors in humans. Further, it displays the SNPs, which are the genetic variations/mutations that are likely linked to a certain disease. FIG. 24 further displays different possible mutations or SNPs that can occur as is immediately apparent to one of ordinary skill in the art.

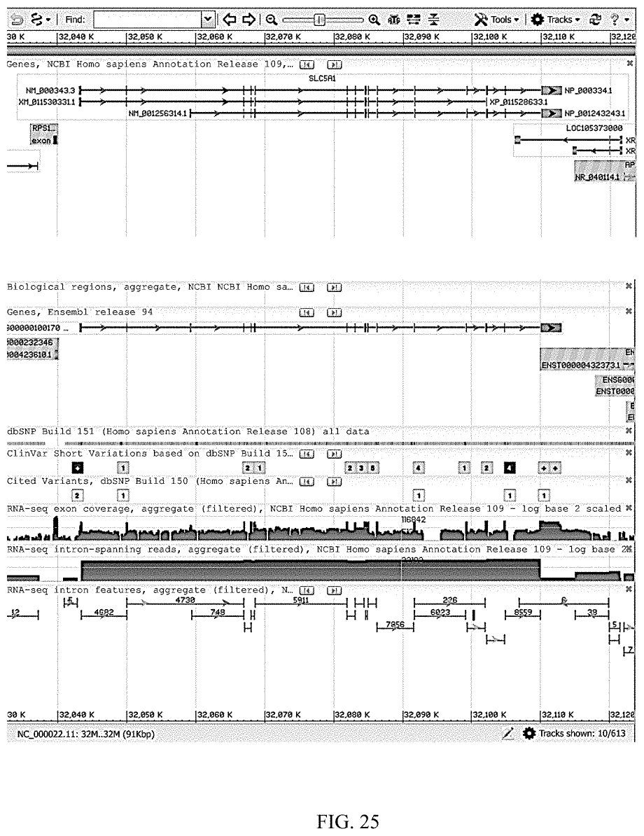

FIG. 25 illustrates the public DNA libraries for SGLT1 receptors in humans. Further, it displays the SNPs that are likely linked to a certain disease. FIG. 25 further displays different possible mutations or SNPs that can occur as is immediately apparent to one of ordinary skill in the art.

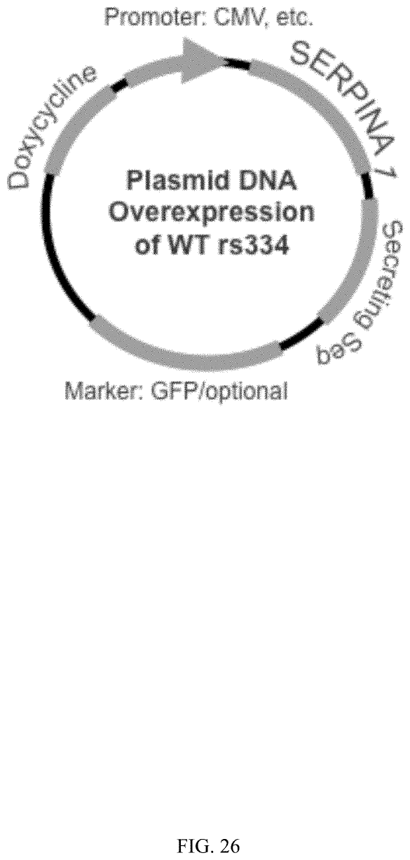

FIG. 26 illustrates a doxycycline-inducible plasmid DNA. In an embodiment the pasmid DNA expresses or overexpresses normal physiological SERPINA1 eliciting the expression of normal levels of AAT according to multiple embodiments of the invention. The plasmid DNA can include CMV promoters and secreting sequences expressing the corrected version of AAT. In another embodiment, the DNA plasmid includes doxycycline, ampicillin, kanamycin, another equivalent agent, or a combination thereof. The plasmid DNA can be loaded in an exosome and the exosome may be autologous or from a universal donor.

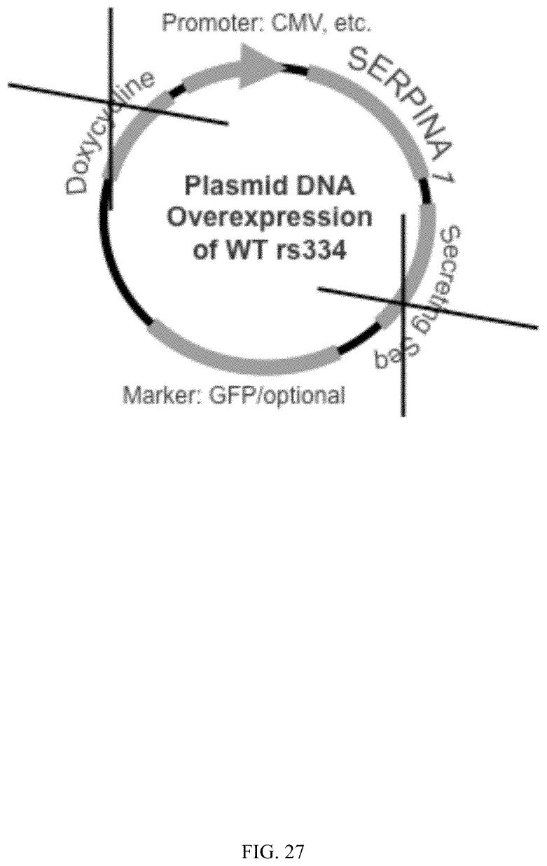

FIG. 27 illustrates a plasmid DNA. In an embodiment, the DNA plasmid expresses or overexpresses normal physiological SERPINA1 eliciting the expression of normal levels of AAT according to multiple embodiments of the invention. The DNA plasmid construct expresses or overexpresses normal SERPINA1 to correct the deficiency of AAT. The plasmid may include doxycycline, ampicillin, kanamycin, a secreting cassette; or a combination thereof. In certain embodiments, the plasmid includes markers such as GFP in preclinical studies. In an embodiment where immunogenicity is not a concern, a GFP cassette is included for human use and human clinical trials.

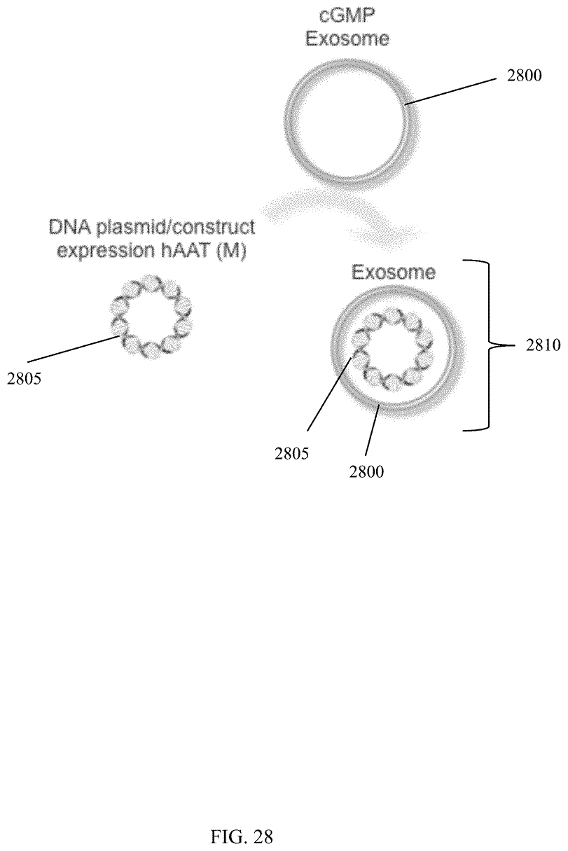

FIG. 28 illustrates a cGMP exosome 2800 loaded with a DNA plasmid 2805. In an embodiment, the DNA plasmid 2805 includes corrected normal human AAT (M allele, SERPINA1). The different methods for isolating the cGMP exosome 2800 have been described above. In one embodiment, an exosome includes a DNA plasmid that corrects SERPINA1 mutations which corrects the expression of normal human AAT (M allele, SERPINA1). The exosome can be loaded with a DNA plasmid alone or in combination with another agent (e.g., siRNA, proteins, antibodies, etc.). Once the exosome 2800 is loaded, the resulting mature exosome 2815 is inspected for cGMP compliance, purity and stability for quality assurance and quality check. As discussed above, the mature exosomes, that have passed the quality check, may undergo an expansion process. The mature exosomes may be diluted and premix into saline for a ready to administer tube. The suspension can be frozen and stored or shipped to a site for use in clinical or preclinical studies and to patients for self-injection of approved-clinical grade mature exosomes.

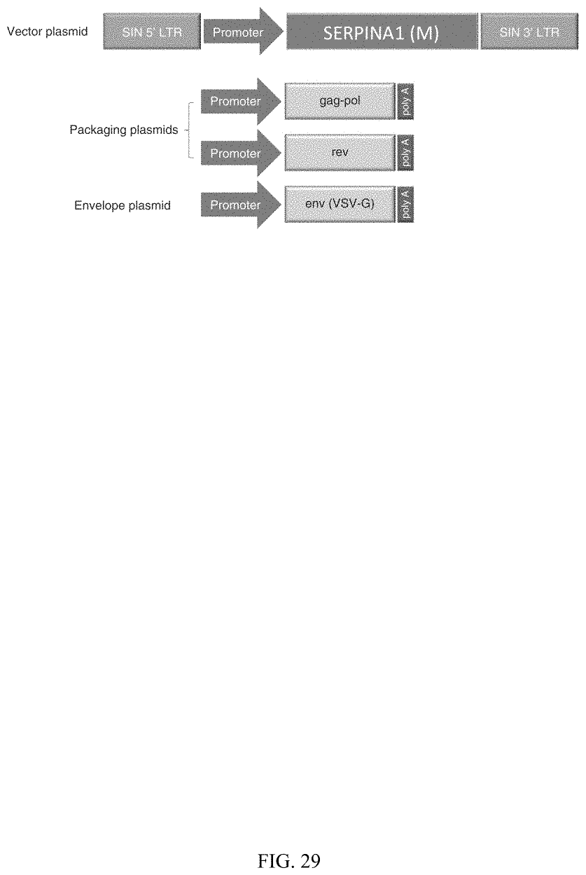

FIG. 29 illustrates lentivirus and/or retrovirus driven expression of SERPINA1 (M) according to multiple embodiments of the invention. The vector can be loaded into an exosome using any of the methods described above. In an embodiment, the exosome is autologous or from a universal donor and is cGMP grade. The vector plasmids can be used to transduce cells and tissues ex vivo or in vivo.

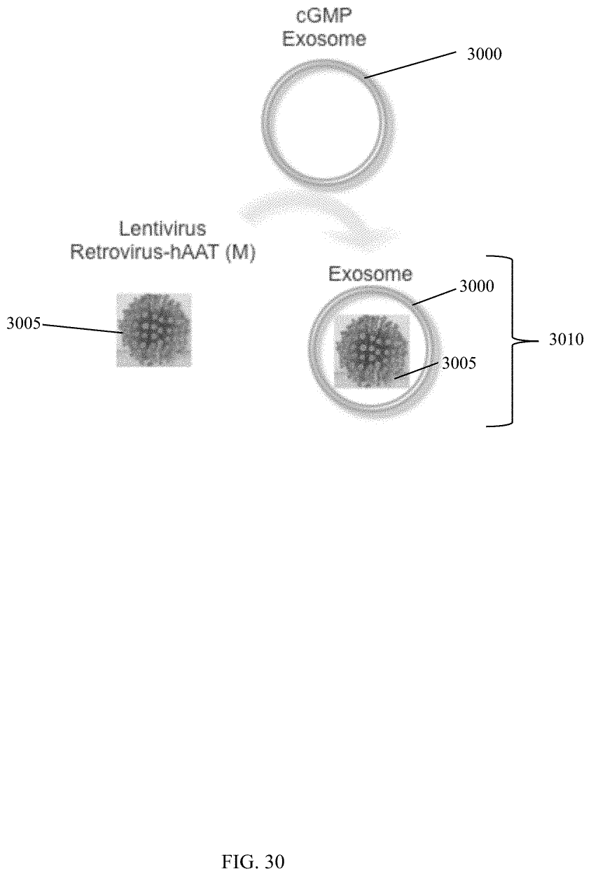

FIG. 30 illustrates a cGMP exosome 3000 loaded with cargo 3005. In an embodiment, the cargocomprises a LTR, lentivirus and/or retrovirus vector 3005 for driving expression of normal human AAT. CMV may be used as a promoter. Other promoters may b used such as, but not limited to, tissue specific promoters (e.g., lung, liver, etc.), SIN, VSV-G, or any combination thereof. The lentivirus and/or retrovirus 3005 is loaded into an exosome 3000 using any of the methods described above. The exosome 3000 can be autologous or from a universal donor. Once the exosome 3000 is loaded with the cargo, the resulting mature exosome 3015 is inspected for cGMP compliance, purity, and stability for quality assurance and quality check. As discussed above, the mature exosomes, that have passed the quality check, may undergo an expansion process. The mature exosomes can be diluted and premix into saline for a ready to administer tube. The suspension can be frozen and stored or shipped to a site for use in clinical or preclinical studies and to patients for self-injection of approved-clinical grade mature exosomes.

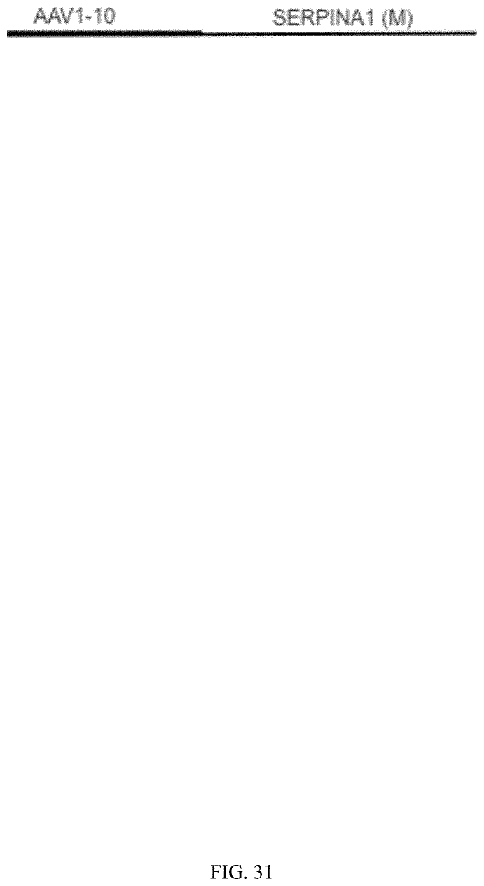

FIG. 31 illustrates an AAV vector (AAV1-10) including the expression sequence for SERPINA1 (M). The vector can be loaded into an exosome using any of the methods described above. The exosome can be autologous or from a universal donor. The AAV1-10 may be utilized to transduce cells and tissues ex vivo or in vivo.

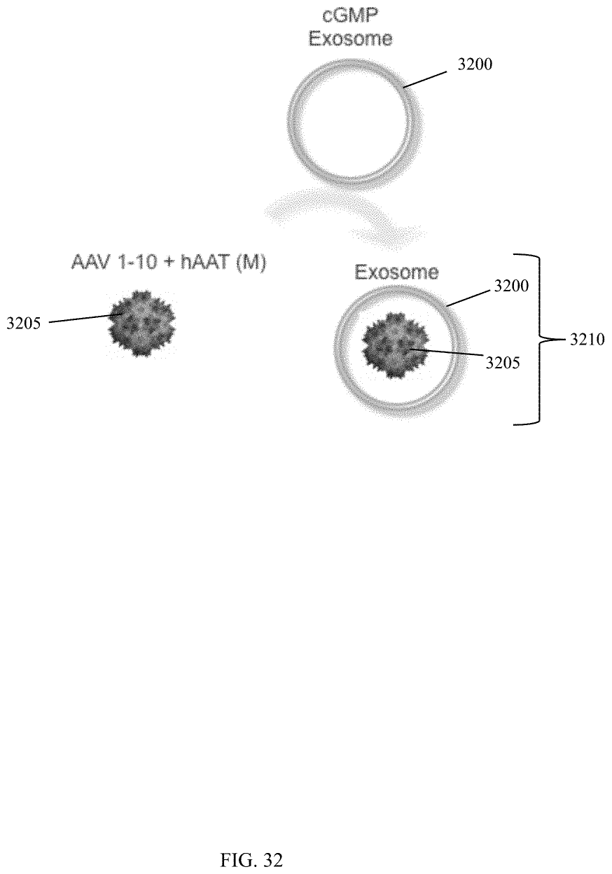

FIG. 32 illustrates a cGMP exosome 3200 loaded with cargo 3205. In an embodiment, the cargo 3205 is an AAV for driving expression of normal human AAT. The exosome 3200 can be autologous or from a universal donor. Once the exosome 3200 is loaded with the cargo, the resulting mature exosome 3215 is inspected for cGMP compliance, purity, and stability for quality assurance and quality check. As discussed above, the mature exosomes, that have passed the quality check, may undergo an expansion process. The mature exosomes can be diluted and premix into saline for a ready to administer tube. The suspension can be frozen and stored or shipped to a site for use in clinical or preclinical studies and to patients for self-injection of approved-clinical grade mature exosomes.

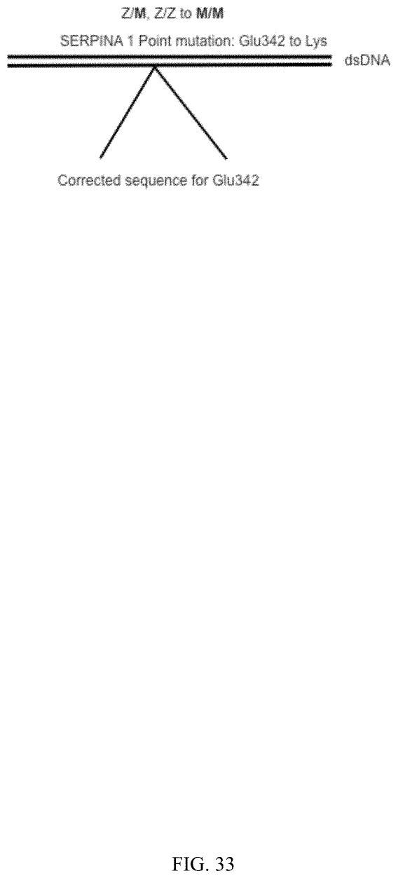

FIG. 33 illustrates insertional mutagenesis to correct the SERPINA1 point mutation glu342 to Lys for the translation of the Z isoform of AAT in one or both alleles of a patient. In an embodiment, insertional mutagenesis can be administered to animals and humans or have the option to do ex vivo bioengineering of particular cell lineages.

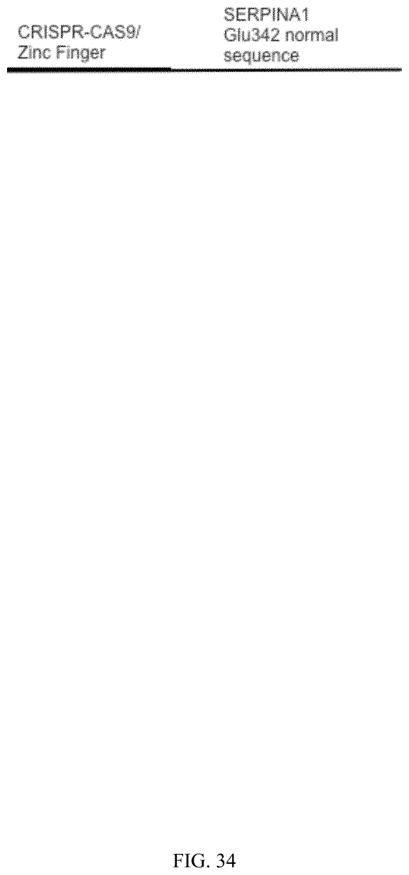

FIG. 34 illustrates gene editing for correcting Lysine to Glutamine in the SERPINA1 gene. In an embodiment, the gene editing nucleases and technology for correction of Lysine to Glutamine (Lys to Glu342) in the SERPINA1 gene include, but are not limited to, CRISPR-CAS9, Zinc finger, TALENS, megalonucleases, or an equivalent, or any combination thereof, etc. In another embodiment, the gene editing technology can include wnt signaling targeting, constructs and engineering for nuclear expressions of relevant main or alternative pathways along with A1ATD and SERPINA1 gene apparent to one of ordinary skill in the art.

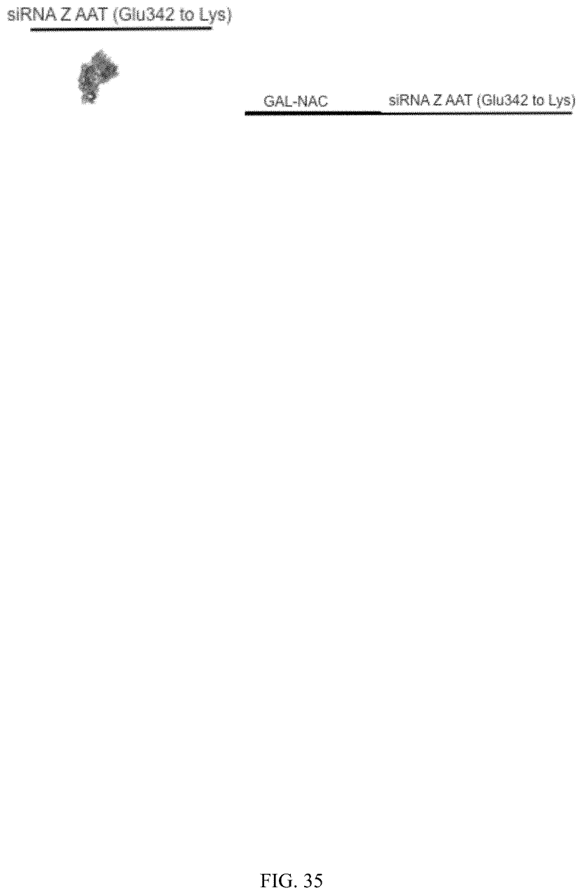

FIG. 35 illustrates siRNA alone or in combination with a GalNAc construct which is used to deplete mutant Z isoform of the SERPINA1 gene. In an embodiment, an exosome comprises siRNA, a GalNAc construct, a DNA plasmid, or a combination thereof. In an embodiment, using a combination of therapies provides for long lasting effects to deplete mutant Z isoform of the SERPINA1 gene to express or overexpress normal physiological ‘M’ AAT. The RNA construct can be used alone as monotherapy in preclinical and clinical trials as well for human use.

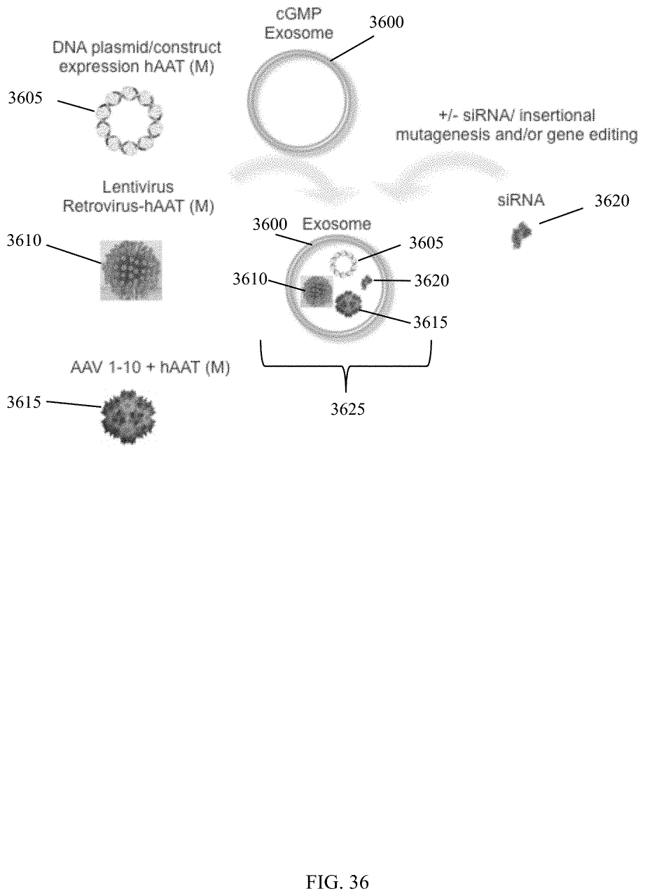

FIG. 36 illustrates a cGMP exosome 3600 loaded with a combination of cargos. The cargos include, a DNA plasmid 3605, a lentivirus and/or retrovirus 3610, an AAV vector 3615, and an siRNA 3620. The combination of therapies provides for long lasting effects to deplete mutant Z isoform of the SERPINA1 gene to express or overexpress normal physiological ‘M’ AAT. The RNA construct can be used alone as monotherapy in preclinical and clinical trials as well for human use. One of the cargos, siRNA 3620, is used to deplete the expression of Z isoform (mutant) of the SERPINA1 gene. siRNA 3620 can be used alone or in combination with a GalNAc construct according to multiple embodiments of the invention.

As discussed above, the mature exosomes 3625, that have passed the quality check, may undergo an expansion process. The mature exosome may be diluted and premixed into saline for a ready to administer tube. Further, the suspension may be frozen and stored or shipped to a site for use in clinical or preclinical studies and to patients for self-injection of approved-clinical grade mature exosomes.

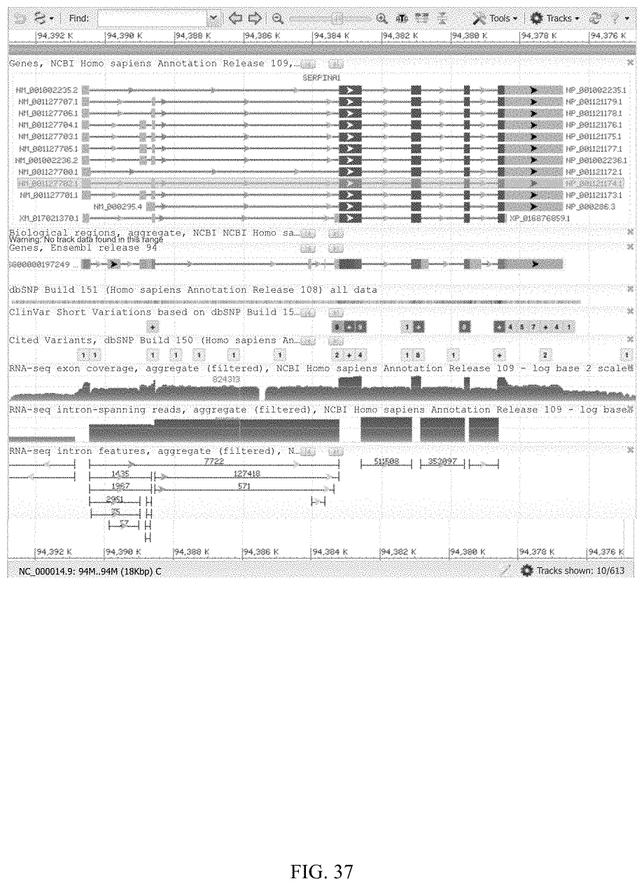

FIG. 37 illustrates SERPINA1 gene (available at https://www.ncbi.nlm.nih.gov/gene/5265).

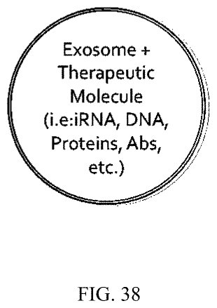

FIG. 38 illustrates an exosome comprising a cargo wherein the cargo includes at least one therapeutic molecule. The therapeutic molecule can be selected from one or a combination of the following group: proteins, peptides, small molecules, RNAi, DNA, proteins, antibodies (Abs), or any other similar agent. In some embodiments, the exosome comprises FAS inhibitors, silencers, or antagonists, including gene silencing using siRNA, siRNA-GalNAc system, editing, and neutralizing antibody generation. The main function of FAS (FASN gene) is to catalyze the synthesis of palmitate from acetyl-CoA and malonyl-CoA, in the presence of NADPH, into long-chain saturated fatty acids. In certain embodiments the plasmid contains a promoter including CMV, liver specific, and others. In certain embodiments, the exosome is loaded with thyroid receptor beta ligands, agonists, or expression vectors. The exosome may comprise Farsenoid X receptors, activators, or modulators by expression vectors, gene editing, or gene modulators. In another embodiment, the exosome comprises uncoupling proteins such as UCP-1 and UCP-2 to overexpress enzymes or the gene itself for applications or indications of obesity and NAFLD/NASH.

In another embodiment, an exosome is loaded with siRNA, another construct, or a combination thereof according to multiple embodiments of the invention. In certain embodiments, the exosome is loaded with GalNAc, siRNA, mRNA, or microRNA for the gene modulation, expression or silencing of CCR2, CCR5, ROS, SNCA, insulin, glucagon, GLP-1, or GIP. In another embodiment, the at least one construct is produced by gene editing nucleases technology, insertional mutagenesis, or a combination thereof.

In multiple embodiments, the exosome can be loaded with at least one cargo. Compounds that may be used as the at least one cargo includes, but is not limited to: 1) Insulin Human Recombinant; 2) Expression Constructs; 3) FGF21 and FGF19 gene silencing and expression constructs; 4) GIP; 5) Agonists, including expression constructs, gene editing or gene silencing; 6) DGAT2 or Diglyceride O-acyltransferase Inhibitors or Silencers, including neutralizing antibody constructs and gene editing; or 7) GLP-1.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

1. A composition for delivering cargo to cytoplasm of a cell, wherein the cargo treats Diabetes Mellitus Type 1 (T1DM) and Diabetes Mellitus Type 2 (T2DM), the composition comprising:
an exosome; and
cargo located within the exosome, wherein the cargo comprises interference RNA (RNAi) that neutralizes sodium-glucose linked transporter (SGLT) active sites.

2. The composition of claim 1, wherein the cargo further comprises small interfering RNA (siRNA), N-Acetylgalactosamine (GalNAc) construct, siRNA-GalNAc construct, or a combination thereof.

3. The composition of claim 1, wherein the cargo further comprises a CRISPR-CAS9 system, a Zinc finger, a single base editor, or a combination thereof.

4. The composition of claim 1, wherein the cargo further comprises a DNA plasmid bioengineered specifically to self-produce monoclonal neutralizing antibodies against SGLT sites, fatty acid synthase (FAS)/FASN, thyroid receptor-b (TR-b)/ligands, uncoupling proteins (UCP-1/UCP-2), C-C chemokine receptor types 2 (CCR2) and 5 (CCR5), reactive oxygen species (ROS), alpha synuclein (SNCA), recombinant insulin/insulin growth factor 1 (hrInsulin/IGF-1), fibroblast growth factor (FGF19/21), Diglyceride acyltransferase or O-acyltransferase (DGAT2), or a combination thereof.

5. The composition of claim 1, wherein the exosome comprises at least one targeting agent, protein epitope, or a combination thereof.

6. The composition of claim 1, wherein the cargo further comprises at least one plasmid, a retrovirus, an adenoassociated virus (AAV), an RNA plasmid, a DNA plasmid, or a combination thereof.

7. The composition of claim 6, wherein the at least one DNA plasmid is designed to overexpress human glucagon like peptide-1 (GLP-1)/glucose-dependent insulinotropic polypeptide (GIP)/glucagon, human FAS/FASN, human TR-b/ligands, human UCP-1/UCP-2, hrInsulin/IGF-1, human FGF19/21, human DGAT2, or a combination thereof.

8. A composition for delivering cargo to cytoplasm of a cell, wherein the cargo treats non-alcoholic fatty liver disease (NAFLD), the composition comprising:
an exosome; and
cargo located within the exosome; wherein the cargo comprises RNAi that neutralizes SGLT active sites.

9. The composition of claim 8, wherein the cargo further comprises siRNA, GalNAc construct, siRNA-GalNAc construct, or a combination thereof.

10. The composition of claim 8, wherein the cargo further comprises a CRISPR-CAS9 system, a Zinc finger, a single base editor, or a combination thereof.

11. The composition of claim 8, wherein the cargo further comprises a DNA plasmid bioengineered specifically to self-produce monoclonal neutralizing antibodies against SGLT sites, FAS/FASN, TR-b/ligands, UCP-1/UCP-2, CCR2CCR5, ROS, SNCA, hrInsulin/IGF-1, FGF19/21, DGAT2, or a combination thereof.

12. The composition of claim 8, wherein the exosome comprises at least one targeting agent, protein epitope, or a combination thereof.

13. The composition of claim 8, wherein the cargo further comprises at least one plasmid, a retrovirus, an AAV, a RNA plasmid, a DNA plasmid, or a combination thereof.

14. The composition of claim 13, wherein the cargo further comprises at least one DNA plasmid designed to overexpress human GLP-1/GIP/glucagon, human FAS/FASN, human TR-b/ligands, human UCP-1/UCP-2, hrInsulin/GF-1, human FGF19/21, human DGAT2, or a combination thereof.

15. A composition for delivering cargo to cytoplasm of a cell, wherein the cargo treats Atherosclerotic Cardiovascular Disease (ASCVD), the composition comprising:
an exosome; and
cargo located within the exosome; wherein the cargo comprises RNAi that neutralizes SGLT active sites.

16. The composition of claim 15, wherein the cargo further comprises siRNA, GalNAc construct, siRNA-GalNAc construct, or a combination thereof.

17. The composition of claim 15, wherein the cargo further comprises a CRISPR-CAS9 system, a Zinc finger, a single base editor, or a combination thereof.

18. The composition of claim 15, wherein the cargo further comprises a DNA plasmid bioengineered specifically to self-produce monoclonal neutralizing antibodies against SGLT sites, FAS/FASN, TR-b/ligands, UCP-1/UCP-2, CCR2CCR5, ROS, SNCA, hrInsulin/IGF-1, FGF19/21, DGAT2, or a combination thereof.

19. The composition of claim 15, wherein the exosome comprises at least one targeting agent, protein epitope, or a combination thereof.

20. The composition of claim 19, wherein the cargo further comprises at least one DNA plasmid designed to overexpress human GLP-1/GIP/glucagon, human FAS/FASN, human TR-b/ligands, human UCP-1/UCP-2, hrInsulin/GF-1, human FGF19/21, human DGAT2, or a combination thereof.
